(19) United States
(12) Patent Application Publication
(10) Pub. No.: US 2014/0031388 A1
(43) Pub. Date: Jan. 30, 2014

(54) AKT INACTIVATION BY TOCOPHERYL DERIVATIVES

(71) Applicants: Ching-Shih Chen, Upper Arlington, OH (US); Dasheng Wang, Dublin, OH (US); Samuel K. Kulp, Hilliard, OH (US)

(72) Inventors: Ching-Shih Chen, Upper Arlington, OH (US); Dasheng Wang, Dublin, OH (US); Samuel K. Kulp, Hilliard, OH (US)

(21) Appl. No.: 13/952,032

(22) Filed: Jul. 26, 2013

Related U.S. Application Data
(60) Provisional application No. 61/675,964, filed on Jul. 26, 2012.

(51) Int. Cl. C07D 311/60; C07D 405/10; C07D 493/10; C07D 215/20; C07D 327/08

(52) U.S. Cl. CPC C07D 311/60; C07D 215/20; C07D 327/08; C07D 493/10; C07D 405/10
USPC 514/311; 514/458; 514/434; 514/454; 514/422; 435/375; 546/179; 549/409; 549/408; 549/404; 549/15; 549/332; 548/525

(57) ABSTRACT

Anticancer compounds according to formula I are described herein.

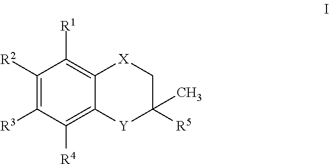

wherein R¹, R², R³ and R⁴ are selected from H, CH₃, OH, SH, OCH₃, NHR′, halogen, CF₃, N-linked pyrrolidine, and SO₂NHR′, or any combination thereof; R⁵ is an alkyl, alkenyl, or alkaryl group including from 4 to 11 carbons, X is selected from CH₂, CHOH, C═O, S═O, O═S═O, and an oxetane ring, Y is selected from CH₂, O, and NH, and R′ is a H, aryl, or a lower alkyl group, or pharmaceutically acceptable salts thereof. The compounds have been shown to facilitate site-specific dephosphorylation of Akt at Ser-473, thereby inactivating Akt and decreasing dysregulation of Akt signaling that can occur in cancer cells.

### CROSS-REFERENCE TO RELATED APPLICATIONS

This application claims priority to U.S. Provisional Patent Application Ser. No. 61/675,964, filed Jul. 26, 2012, which is incorporated herein by reference.

### STATEMENT ON FEDERALLY FUNDED RESEARCH

This invention was funded, at least in part, by National Institutes of Health Grant NIH-R01CA11250 and GRT00019777. The federal government has certain rights in this invention.

### BACKGROUND

Although the Selenium and Vitamin E Cancer Prevention Trial (SELECT) failed to demonstrate the chemopreventive effect of α-tocopherol in prostate cancer, considerable interest still exists in evaluating the anti-tumorigenic effects of γ- and other forms of tocopherol in light of their superior anti-inflammatory and antitumor efficacies. Ju et al., Carcinogenesis 31, 533 (2010). For example, γ-tocopherol exhibits greater potency than α-tocopherol in suppressing prostate cancer cell proliferation and carcinogen-induced transformation of murine fibroblasts. Gysin et al., FASEB J 16, 1952 (2002); Cooney et al., Proc Natl Acad Sci USA 90, 1771 (1993).

From a translational perspective, a major impediment to the clinical development of vitamin E for cancer prevention is a lack of understanding of the molecular target by which tocopherols mediate antiproliferative effects. Evidence has implicated various mechanisms by which tocopherols perturb cancer cell function and survival independent of antioxidant properties. R. Brigelius-Flohe, Free Radic Biol Med 46, 543 (2009). Among these, dephosphorylation of Akt by tocopherols, though at high concentrations, is especially noteworthy in light of the role of Akt signaling in mediating cancer cell survival. Kempna et al., J Biol Chem 279, 50700 (2004); Lee et al., Clin Cancer Res 15, 4242 (2009). However, there remains a need to better understand the mechanism by which vitamin E derivatives provide an anticancer effect, and to identify derivatives that are particularly effective.

### SUMMARY OF THE INVENTION

The mechanism by which α- and γ-tocopherol facilitate the selective dephosphorylation of the kinase Akt at Ser473 was investigated. The inventors showed that this site-specific Akt dephosphorylation was mediated through the pleckstrin homology (PH) domain-dependent recruitment to the plasma membrane of Akt and PHLPP (PH domain leucine-rich repeat protein phosphatase, isoform 1), a phosphatase that dephosphorylates Akt at Ser473. The ability of α- and γ-tocopherol to induce PHLPP-mediated Akt inhibition established PHLPP as a “druggable” target. The tocopherols were structurally optimized to obtain derivatives with greater in vitro potency and in vivo tumor-suppressive activity in two prostate xenograft tumor models. Binding affinities for the PH domains of Akt and PHLPP were greater than for other PH domain-containing proteins, which may underlie the preferential membrane recruitment of these proteins. Molecular modeling revealed the structural determinants of the interaction with the PH domain of Akt that may inform strategies for continued structural optimization. These findings describe a mechanism by which tocopherols facilitate the dephosphorylation of Akt at Ser473, thereby providing insights into the mode of antitumor action of tocopherols and a rationale for the translational development of tocopherols into novel PH domain-targeted Akt inhibitors.

In one aspect, the invention provides a compound according to formula I:

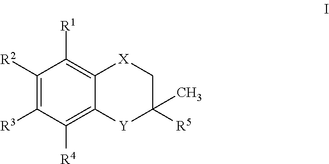

wherein R¹, R², R³ and R⁴ are selected from H, CH₃, OH, SH, OCH₃, NHR′, halogen, CF₃, N-linked pyrrolidine, and SO₂NHR′, or any combination thereof; R⁵ is an alkyl, alkenyl, or alkaryl group including from 4 to 11 carbons, X is selected from CH₂, CHOH, C═O, S═O, O═S═O, and an oxetane ring, Y is selected from CH₂, O, and NH, and R′ is a H, aryl, or a lower alkyl group, or pharmaceutically acceptable salts thereof. In another aspect, a method of treating cancer in a subject in need thereof, by administering to the subject a therapeutically effective amount of a compound according to Formula I is provided. In a further aspect, a method of inactivating Akt in a tumor cell by contacting the tumor cell with an effective amount of a compound according to formula I is provided.

### BRIEF DESCRIPTION OF THE FIGURES

The present invention may be more readily understood by reference to the following drawings, wherein:

FIG. 1 provides graphs and images showing that tocopherols induce apoptosis and the Ser⁴⁷³-specific dephosphorylation and membrane localization of Akt. (A) Chemical structures of α- and γ-tocopherol (upper panel) and their antiproliferative effects (lower panel) in prostate cancer cell lines LNCaP and PC-3 cultured in serum-free medium as determined by MTT assays. Points, means; bars, ±S.D. (n=6 replicates; data from a representative experiment). α,α-tocopherol; γ,γ-tocopherol. (B) Flow cytometric analysis of apoptosis in LNCaP cells measured by Annexin V and PI staining after exposure to α-tocopherol (α-Toco) and γ-tocopherol (γ-Toco) in serum-free medium (N=3 experiments). (C) Western blot analysis of the effects of α- and γ-tocopherol on the phosphorylation of Ser⁴⁷³ and Thr³⁰⁸ Akt (p-Ser⁴⁷³-Akt and p-Thr³⁰⁸-Akt) in LNCaP cells. A representative Western blot and relative phosphorylation of Akt at Ser⁴⁷³ and Thr³⁰⁸ from 4 independent experiments are shown. Signals from phosphorylated Akt were first normalized to that of total Akt, and then to that of β-actin. *, P<0.05 compared with the DMSO control (Kruskal-Wallis). (D) Plasma membrane localization of Akt in LNCaP cells exposed to α- and γ-tocopherol in serum-free medium. Left, Immunofluorescence staining of Akt and DAPI (4′,6-diamidino-2-phenylindole)-stained nuclei. Scale bars, 40 μm. Arrows indicate cells selected for analysis of fluorescent intensity through a cross-sectional plane (center, yellow line). Right, Three-dimensional surface plots of whole cell fluorescence intensities. Two-dimensional histograms show cross-sectional fluorescence intensities. (E) Percentages of cells with Akt membrane localization in response to α- and γ-tocopherol versus DMSO (P<0.05, one-way ANOVA). 73-142 cells were analyzed for each treatment condition in three independent experiments.

FIG. 2 provides graphs and images showing that tocopherols facilitate the co-localization of Akt and PHLPP at the plasma membrane. (A) Plasma membrane localization of PHLPP in LNCaP cells exposed to α- and γ-tocopherol in serum-free medium. Left column, Immunofluorescence staining of PHLPP1, including total PHLPP and DAPI-stained nuclei. Arrows, cells selected for analysis of fluorescence intensity. Left center column, Cells selected for analysis of fluorescent intensity. Light lines, cross-sectional planes of analysis. Right center column, Three-dimensional surface plots of whole cell fluorescence intensities. Right column, Two-dimensional histograms of cross-sectional fluorescence intensities. Scale bars, 40 μm. (B) Percentages of cells with membrane localization of PHLPP1 in response to α- and γ-tocopherol versus DMSO (*P=0.058?, Kruskal-Wallis). A total of 15-40 cells were analyzed for each treatment condition in three independent experiments. (C) Representative Western blot of the phosphorylation of Ser⁴⁷³ in Akt, Akt, PHLPP1, and PHLPP2 in the membrane (left) and cytoplasmic (right) fractions of LNCaP cells treated with α-tocopherol (α-Toco) and γ-tocopherol (γ-Toco). (D) Co-immunoprecipitation of Akt-PHLPP complexes from the membrane fractions in (C). Upper panel, Immunoprecipitation (IP) was performed using anti-Akt antibody, followed by Western blotting of PHLPP and Akt. A representative immunoblot is shown. Lower panel, Association between PHLPP and Akt in the membranes of treated LNCaP cells. Amounts of immunoblotted proteins were quantitated by densitometry. Signals from immunoprecipitated PHLPP were normalized to that of total Akt. Values, means; bars, ±S.D. (N=3 experiments. *, P<0.05 compared to DMSO control (one-way ANOVA).

FIG. 3 provides graphs and images showing that truncation of the aliphatic side chains of α- and γ-tocopherol enhances ability to induce apoptotic death through Akt inactivation. (A) The chemical structures of α-VE5 and γ-VE5, which are truncated derivatives of α-tocopherol and γ-tocopherol, respectively, (upper panel) and antiproliferative effects of α- and γ-VE5 in LNCaP and PC-3 cells as determined by MTT assays following exposure in serum-free medium (lower panel). Points, means; bars, ±S.D. (n=6 replicates; data from a representative experiment), α,α-VE5; γ,γ-VE5. (B) Flow cytometric analysis of apoptosis in LNCaP cells (Annexin V/PI staining) after exposure to α- and γ-VE5 in serum-free medium (N=three experiments). (C) Effects of α- and γ-VE5 on the phosphorylation of Ser⁴⁷³ and Thr³⁰⁸ in LNCaP cells. Left panel, Representative Western blot of phosphorylation at Ser⁴⁷³ and Thr³⁰⁸ in Akt. Right panel, Relative phosphorylation of Ser⁴⁷³ and ³⁰⁸Thr in Akt. Amounts of immunoblotted proteins were quantitated by densitometry. Signals from phosphorylated Akt were first normalized to that of total Akt, and then to that of β-actin. Values, means; bars, ±S.D. (N=3 experiments; replicate data are shown in FIG. S3A). *, P<0.05 (Kruskal-Wallis). (D) Protective effect of the ectopic expression of constitutively active Akt (CA-Akt) against the antiproliferative activities of α- and γ-VE5 in LNCaP cells. Control cells were transfected with the empty vector (pcDNA). Points, means; bars, ±S.D. (n=6 replicates; data from a representative experiment).

FIG. 4 provides graphs and images showing that α-VE5 and γ-VE5 facilitate the recruitment of Akt and PHLPP to the plasma membrane. Plasma membrane localization of (A) Akt and (B) PHLPP in LNCaP cells exposed to α- and γ-VE5. Membrane localization of Akt was observed in 65.0±8.9% and 71.4±2.5% of cells treated with α- and γ-VE5, respectively, compared to 38.0±5.7% in DMSO-treated cells (P<0.05, one-way ANOVA; a total of 49-101 cells were analyzed for each treatment condition in three independent experiments, for which the replicate data are shown in FIG. S4A). Left columns, Immunofluorescence staining of Akt or PHLPP. Arrows, cells selected for analysis of fluorescence intensity, including total Akt or PHLPP and DAPI-stained nuclei. Left center columns, Cells selected for analysis of fluorescence intensity. Light lines, cross-sectional planes of analysis. Right center columns, Three-dimensional surface plots of whole cell fluorescence intensities. Right columns, Two-dimensional histograms of cross-sectional fluorescence intensities. Scale bars, 40 μm. (C) Percentage of cells with membrane localization of Akt or PHLPP in response to α- and γ-VE5 versus DMSO-treated cells (*P<0.05, Kruskal-Wallis). A total of 32-47 cells were analyzed for each treatment condition in three independent experiments. (D) Representative Western blot of the phosphorylation of Ser⁴⁷³ in Akt, Akt, PHLPP1, and PHLPP2 in the membrane (left) and cytoplasmic (right) fractions of LNCaP cells treated with α- and γ-VE5. (E) Co-immunoprecipitation of Akt-PHLPP complexes from the membrane fractions described above in (D). Left panel, Immunoprecipitation (IP) was performed using anti-Akt antibody, followed by Western blotting of PHLPP1, PHLPP2, and Akt. A representative immunoblot is shown. Right panel, Relative association of PHLPP1 or PHLPP2 with Akt in the membranes of treated LNCaP cells. Amounts of immunoblotted proteins were quantitated by densitometry. Signals from immunoprecipitated PHLPP1 and PHLPP2 were normalized to that of total Akt. Values, means; bars, ±S.D. (N=3 experiments). *, P<0.05 compared to DMSO control (Welch's ANOVA).

FIG. 5 provides graphs and images showing α-VE5 and γ-VE5-induced co-localization of Akt and PHLPP1 to the non-raft domains of the plasma membrane, essential role of PHLPP in VE5-induced Akt dephosphorylation, and in vivo tumor-suppressive activity of γ-VE5. (A) Co-localization of Akt and PHLPP1 to non-raft membrane domains in LNCaP cells treated with α- and γ-VE5. Upper panels, Representative Western blot of PHLPP1, Akt phosphorylated at Ser⁴⁷³, Akt, PDK1 and flotillin-2 in cell membrane subfractions. Lipid raft-containing fractions were identified by the presence of flotillin-2 (fractions 3-5). Lower left panel, Representative Western blot of PHLPP1 and Akt phosphorylated at Ser⁴⁷³ in the non-raft fractions 10-12. Lower right, Relative amounts of PHLPP1 and Akt phosphorylated at Thr⁴⁷³ in the non-raft fractions 10-12. Amounts of immunoblotted proteins were quantitated by densitometry. Signals from PHLPP1 and Akt phosphorylated at Ser⁴⁷³ were normalized to that of PDK1, the cellular distribution of which was not disturbed by α- or γ-VE5 (FIG. 6B). Values, means; bars, ±S.D. (N=3 experiments; replicate data are shown in FIG. S5A). *, P<0.05 compared to respective DMSO control (Kruskal-Wallis). (B) Quantitative analysis of cholesterol content in individual membrane subfractions from cells described in (A). Values, means; bars, ±S.D. (N=3 experiments). *, P<0.05 compared to the corresponding fraction in DMSO control (one-way ANOVA) (C) Protective effect of siRNA-mediated PHLPP1 knockdown against the suppressive activities of γ-VE5 on cell viability [left; Points, means; bars, ±S.D. (n=6)], and phosphorylation of Ser⁴⁷³ in Akt (middle, representative Western blot is shown) in LNCaP cells. Right, Relative phosphorylation of Ser⁴⁷³ in Akt. Amounts of immunoblotted proteins were quantitated by densitometry. Signal from phosphorylated Ser⁴⁷³ was first normalized to that of total Akt, and then to that of β-actin. Values, means; bars, ±S.D. (N=3 experiments; replicate data are shown in FIG. S5B). *, P<0.05 compared to respective DMSO control (Welch's ANOVA). (D) γ-VE5 suppresses PC-3-luc xenograft tumor growth in vivo. Athymic nude mice bearing established subcutaneous PC-3-luc xenograft tumors were treated with vehicle or γ-VE5. Tumor burdens were measured weekly by bioluminescent imaging. Left, Effect of γ-VE5 on the growth of PC-3-luc tumors as represented by relative bioluminescence. Inset, representative tumors from vehicle- and γ-VE5-treated mice. Points, means; bars, ±S.D. (n=7 mice). Middle, Western blot analysis of the phosphorylation status of Akt at Ser⁴⁷³ compared to Thr³⁰⁸, MDM2, and IKKα in five representative PC-3 tumors from each group of mice. Right, Relative intratumoral phosphorylation of Ser⁴⁷³ compared to that of Thr³⁰⁸ in Akt. Amounts of immunoblotted proteins were quantitated by densitometry. Signals from phosphorylated Ser⁴⁷³ and Thr³⁰⁸ were first normalized to that of total Akt, and then to that of β-actin. Values, means; bars, ±S.D. (n=5 tumors). *, P<0.05 compared to vehicle control (Mann-Whitney U).

FIG. 6 provides images showing membrane recruitment of Akt and PHLPP by tocopherols and VE5 compounds is PH domain-dependent. (A) Subcellular localization of green fluorescent protein (GFP)-tagged full length wild-type Akt (GFP-Akt), PH domain of Akt (GFP-PH^{Akt}), and PH domain-deleted Akt (GFP-ΔPH-Akt) in LNCaP cells treated with α-VE5 in serum-free medium. Scale bars, 40 μm. Additional images from three independent experiments are shown in FIG. S6A. (B) Subcellular localization of GFP-PHLPP, GFP-PH^{PHLPP}, and GFP-ΔPH-PHLPP in LNCaP cells treated as described in (A). Scale bars, 40 μm. (C) Subcellular distribution of PH-domain containing kinases PDK-1 (left), ILK (middle), and BTK (right) in LNCaP cells treated with tocopherols and VE5 compounds. Scale bars, 40 μm.

FIG. 7 provides images showing the molecular modeling of the binding of α- and γ-VE5 with the Akt-PH domain. (A) A model for the docking of γ-VE5 into the VL2 loop of the PH domain of Akt. Binding of phosphatidylinositol (3,4,5)-triphosphate (PIP₃) is also represented. (B) Modeled interactions of γ-VE5 (left) and α-VE5 (right) with amino acid residues of the VL2 domain of the Akt PH domain. VE5 compounds and hydrogen bonding (dashed line) are indicated. (C) Diagram depicting the mechanism of γ-tocopherol-mediated dephosphorylation of Ser⁴⁷³ in Akt in cancer cells (right) compared to that of PIP₃-mediated Akt activation (left). PIP₃, phosphatidylinositol (3,4,5)-triphosphate; PH, pleckstrin homology.

FIG. 8 provides a scheme depicting the synthesis of α- and γ-VE5. Reagents and Reaction conditions: a) Wittig reaction, Ph₃PCH₂CH₂(CH₃)₂Br, Li-BTSA, THF, 0° C. to r.t.; b) 10% Pd/C, H₂, Ethyl acetate, 35 psi, r.t.; c) Br², Hexane, r.t. 3 h then Ac₂O, AcOH, H₂SO₄, r.t.; d) NMPO, acetonitrile, r.t.; e) NH₂SO₃H, NaClO₂, rt; f) Con. HCl, MeOH reflux; g) Heat 170° C.

FIG. 9 provides a scheme depicting the synthesis of compounds 3, 6-15. Reagents and Reaction conditions: a) pyrrolidine, toluene, reflux; b) Zn/HCl, MeOH, rt.

FIG. 10 provides (A) a scheme showing the synthesis and structure of compound 1 and (b) the dose-dependent effect of compound 1 on Ser-473 Akt phosphorylation.

FIG. 11 provides images and schemes showing (A) Global docking of compound 7 into the Akt PH domain. (B) Diagrams depicting the mode of binding of compound 7 versus those of its isosteric derivatives 14 and 15. ΔΔG denotes predicted binding free energy. (C) List of compounds to be generated from the proposed isosteric replacement.

### DETAILED DESCRIPTION OF THE INVENTION

The inventors have developed a new paradigm of α/γ-tocopherol as PHLPP1-targeted Akt inhibitors. Among the reported antiproliferative mechanisms, the effect of high concentrations of tocopherols on Akt dephosphorylation is especially noteworthy in light of the important role of Akt signaling in promoting cancer cell survival. The data indicate that α-tocopherol and, to a greater extent, γ-tocopherol facilitate the site-specific dephosphorylation of Akt at Ser-473 with activities paralleling their respective antiproliferative potencies in prostate cancer cells. Moreover, the inventors have obtained evidence that this selective Akt dephosphorylation is attributable to a heretofore unreported mechanism whereby α- and γ-tocopherol facilitate the co-recruitment of Akt and PHLPP1 to the plasma membrane through PH domain recognition. The ability of α- and γ-tocopherol to induce PHLPP1-mediated Akt inhibition demonstrates that PHLPP is a “druggable” target.

PHLPP consists of two isoforms, PHLPP1 and PHLPP2, which are important negative regulators of Akt through Ser-473 dephosphorylation, of which the tumor suppressor role of PHLPP1 is especially noteworthy. Evidence indicates that PHLPP1 and PTEN form a tumor suppressor network that modulates the PI3K-Akt signaling axis, which is often dysregulated in the course of tumorigenesis. For example, PHLPP1 mediates AR-induced Akt inhibition, indicating its involvement in the intricate feedback crosstalk between PI3K/Akt and AR signaling. Moreover, deletion of phlpp1 caused neoplasia and cancer in prostates of Pten-deficient mice, which suggests its role in counteracting the effect of loss of PTEN function on Akt activation.

The ability of tocopherols to induce this PHLPP1-mediated Akt inactivation provides a mechanistic basis for the structure-based lead optimization of tocopherols to develop potent Akt inhibitors. The proof-of-principle of this premise was provided by α- and γ-VE5, which are side chain-truncated derivatives of α- and γ-tocopherol, respectively, with 50-fold higher potencies relative to their parent compounds in facilitating Ser-473-Akt dephosphorylation through PHLPP1 co-recruitment and in suppressing the viability of prostate cancer cells. A variety of additional tocopheryl derivatives showing further increased activity are described herein.

### DEFINITIONS

The terminology as set forth herein is for description of the embodiments only and should not be construed as limiting of the invention as a whole. As used in the description of the invention and the appended claims, the singular forms “a”, “an”, and “the” are inclusive of their plural forms, unless contraindicated by the context surrounding such.

As used herein, the term “organic group” is used to mean a hydrocarbon group that is classified as an aliphatic group, cyclic group, or combination of aliphatic and cyclic groups (e.g., alkaryl and aralkyl groups). In the context of the present invention, suitable organic groups for tocopheryls of this invention are those that do not interfere with the energy restriction activity of the tocopheryls. In the context of the present invention, the term “aliphatic group” means a saturated or unsaturated linear or branched hydrocarbon group. This term is used to encompass alkyl, alkenyl, and alkynyl groups, for example.

As used herein, the terms “alkyl”, “alkenyl”, and the prefix “alk-” are inclusive of straight chain groups and branched chain groups. Unless otherwise specified, these groups contain from 1 to 20 carbon atoms, with alkenyl groups containing from 2 to 20 carbon atoms. In some embodiments, these groups have a total of at most 10 carbon atoms, at most 8 carbon atoms, at most 6 carbon atoms, or at most 4 carbon atoms. Alkyl groups including 4 or fewer carbon atoms can also be referred to as lower alkyl groups.

Cycloalkyl, as used herein, refers to an alkyl group (i.e., an alkyl, alkenyl, or alkynyl group) that forms a ring structure. Cyclic groups can be monocyclic or polycyclic and preferably have from 3 to 10 ring carbon atoms. A cycloalkyl group can be attached to the main structure via an alkyl group including 4 or less carbon atoms. Exemplary cyclic groups include cyclopropyl, cyclopropylmethyl, cyclopentyl, cyclohexyl, adamantyl, and substituted and unsubstituted bornyl, norbornyl, and norbornenyl.

Unless otherwise specified, “alkylene” and “alkenylene” are the divalent forms of the “alkyl” and “alkenyl” groups defined above. The terms, “alkylenyl” and “alkenylenyl” are used when “alkylene” and “alkenylene”, respectively, are substituted. For example, an arylalkylenyl group comprises an alkylene moiety to which an aryl group is attached.

The term “haloalkyl” is inclusive of groups that are substituted by one or more halogen atoms, including perfluorinated groups. This is also true of other groups that include the prefix “halo-”. Examples of suitable haloalkyl groups are chloromethyl, trifluoromethyl, and the like. Halo moieties include chlorine, bromine, fluorine, and iodine.

The term “aryl” as used herein includes carbocyclic aromatic rings or ring systems. Examples of aryl groups include phenyl, naphthyl, biphenyl, fluorenyl and indenyl. Aryl groups may be substituted or unsubstituted.

Unless otherwise indicated, the term “heteroatom” refers to the atoms O, S, or N. The term “heteroaryl” includes aromatic rings or ring systems that contain at least one ring heteroatom (e.g., O, S, N). In some embodiments, the term “heteroaryl” includes a ring or ring system that contains 2 to 12 carbon atoms, 1 to 3 rings, 1 to 4 heteroatoms, and O, S, and/or N as the heteroatoms. Suitable heteroaryl groups include furyl, thienyl, pyridyl, quinolinyl, isoquinolinyl, indolyl, isoindolyl, triazolyl, pyrrolyl, tetrazolyl, imidazolyl, pyrazolyl, oxazolyl, thiazolyl, benzofuranyl, benzothiophenyl, carbazolyl, benzoxazolyl, pyrimidinyl, benzimidazolyl, quinoxalinyl, benzothiazolyl, naphthyridinyl, isoxazolyl, isothiazolyl, purinyl, quinazolinyl, pyrazinyl, 1-oxidopyridyl, pyridazinyl, triazinyl, tetrazinyl, oxadiazolyl, thiadiazolyl, and so on.

When a group is present more than once in any formula or scheme described herein, each group (or substituent) is independently selected, whether explicitly stated or not. For example, for the formula —C(O)—NR₂ each R group is independently selected.

As a means of simplifying the discussion and the recitation of certain terminology used throughout this application, the terms “group” and “moiety” are used to differentiate between chemical species that allow for substitution or that may be substituted and those that do not so allow for substitution or may not be so substituted. Thus, when the term “group” is used to describe a chemical substituent, the described chemical material includes the unsubstituted group and that group with nonperoxidic O, N, S, Si, or F atoms, for example, in the chain as well as carbonyl groups or other conventional substituents. Where the term “moiety” is used to describe a chemical compound or substituent, only an unsubstituted chemical material is intended to be included. For example, the phrase “alkyl group” is intended to include not only pure open chain saturated hydrocarbon alkyl substituents, such as methyl, ethyl, propyl, tert-butyl, and the like, but also alkyl substituents bearing further substituents known in the art, such as hydroxy, alkoxy, alkylsulfonyl, halogen atoms, cyano, nitro, amino, carboxyl, etc. Thus, “alkyl group” includes ether groups, haloalkyls, nitroalkyls, carboxyalkyls, hydroxyalkyls, sulfoalkyls, etc. On the other hand, the phrase “alkyl moiety” is limited to the inclusion of only pure open chain saturated hydrocarbon alkyl substituents, such as methyl, ethyl, propyl, tert-butyl, and the like.

The invention is inclusive of the compounds described herein in any of their pharmaceutically acceptable forms, including isomers (e.g., diastereomers and enantiomers), tautomers, salts, solvates, polymorphs, prodrugs, and in liposome/nanoparticle preparations. In particular, if a compound is optically active, the invention specifically includes each of the compound's enantiomers as well as racemic mixtures of the enantiomers. It should be understood that the term “compound” includes any or all of such forms, whether explicitly stated or not (although at times, “salts” are explicitly stated). In particular, in some embodiments, the invention may be directed to the 2-(R) form of any of the tocopheryl derivatives described herein.

The term tocopheryl derivatives, as used herein, is a shorthand for the tocopheryl compounds of the invention, as described by the formulas provided herein; and is not meant to encompass all possible compounds that might be characterized as being a derivative of tocopheryl by one skilled in the art.

Treat”, “treating”, and “treatment”, etc., as used herein, refer to any action providing a benefit to a subject at risk for or afflicted with a condition or disease such as cancer, including improvement in the condition through lessening or suppression of at least one symptom, delay in progression of the disease, prevention or delay in the onset of the disease, etc. The subject may be at risk due to exposure to carcinogenic agents, being genetically predisposed to disorders characterized by unwanted, rapid cell proliferation, and so on.

“Pharmaceutically acceptable” as used herein means that the compound or composition is suitable for administration to a subject for the methods described herein, without unduly deleterious side effects in light of the severity of the disease and necessity of the treatment.

The terms “therapeutically effective” and “pharmacologically effective” are intended to qualify the amount of a compound which will achieve the goal of improvement in disease severity and the frequency of incidence over treatment of each compound by itself, while avoiding adverse side effects typically associated with alternative therapies. The term “effective amount” is intended to qualify the amount of a compound which will achieve an effect which may lead to a therapeutic effect, but which is not itself a therapeutic effect, such as inhibition or activation of a particular enzyme.

### Tocopheryl Derivatives

A variety of tocopheryl derivatives are described herein. In one aspect, the invention provides a number of tocopheryl derivative compounds according to formula I:

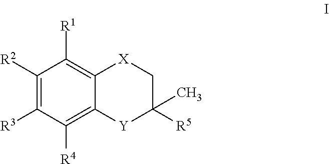

wherein R¹, R², R³ and R⁴ are selected from H, CH₃, OH, SH, OCH₃, NHR′, halogen, CF₃, linked pyrrolidine, and SO₂NHR′, or any combination thereof; R⁵ is an alkyl, alkenyl, or alkaryl group including from 4 to 11 carbons, X is selected from CH₂, CHOH, C═O, S═O, O═S═O, and an oxetane ring, Y is selected from CH₂, O, and NH, and R′ is a H, aryl, or a lower alkyl group, or pharmaceutically acceptable salts thereof. With regard to X, it should further be added that in the case of C═O, S═O, or O═S═O, attachment to the ring occurs at either the carbon or the sulfur atom, while in the case of an oxetane ring, attachment occurs at C-2 of the oxetane ring, as shown in compound 26 (8-bromo-2-methyl-2-(4-methylpentyl)-spiro[chroman-4,3′-oxetane]-6-sulfonamide) of example 2 described herein.

A number of embodiments of the invention are directed to subsets of compounds within formula I. For example, in some embodiments, R⁵ is an alkyl group having from 4 to 6 carbons, while in other embodiments R⁵ is an alkyl, alkenyl, or alkaryl group having from 7 to 11 carbons. In other embodiments, Y is an O (oxygen), and in further embodiments, X is selected from C═O, S═O, and O═S═O, or in some cases X is C═O.

In other embodiments, various combinations of substituents can be provided at R¹, R², R³, and R⁴ along the phenyl ring of formula I. For example, in some embodiments R¹ and R³ are H, while in further embodiments R² is OH.

In other embodiments, a variety of the substituents are further defined, or particular compounds within formula I may be specified. For example, in some embodiments, R² is OH, X is C═O, and Y is O. In other embodiments, the compound is selected from the following structures:

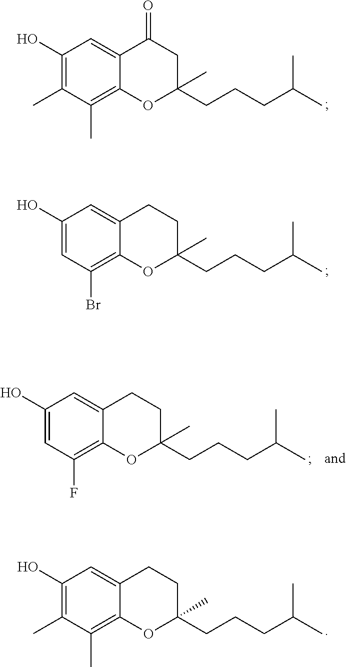 [SMILES: Cc1c(O)cc2c(c1C)OC(C)(CCCC(C)C)CC2=O ; CC(C)CCCC1(C)CCc2cc(O)cc(Br)c2O1 ; CC(C)CCCC1(C)CCc2cc(O)cc(F)c2O1 ; Cc1c(O)cc2c(c1C)O[C@](C)(CCCC(C)C)CC2]

### Cancer Treatment Using Tocopheryl Derivatives

Another aspect of the invention provides a method of treating cancer in a subject in need thereof by administering to the subject a therapeutically effective amount of a compound according to Formula I:

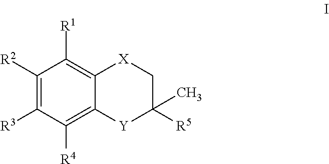

wherein R¹, R², R³ and R⁴ are selected from H, CH₃, OH, SH, OCH₃, NHR′, halogen, CF₃, N-linked pyrrolidine, and SO₂NHR′, or any combination thereof; R⁵ is an alkyl, alkenyl, or alkaryl group including from 4 to 11 carbons, X is selected from CH₂, CHOH, C═O, S═O, O═S═O, and an oxetane ring, Y is selected from CH₂, O, and NH, and R′ is a H, aryl, or a lower alkyl group, or a pharmaceutically acceptable salt thereof.

In other embodiments, the compounds being administered can be selected from any of the particular embodiments described for the tocopheryl derivative compounds of Formula I provided herein. For example, in some embodiments, R² of the compound of formula I is OH. In other embodiments, X of the compound of formula I is selected from C═O, S═O, and O═S═O. In further embodiments, the compound of formula I is further defined such that R² is OH, X is C═O, and Y is O.

A subject, as defined herein, is an animal, preferably a mammal such as a domesticated farm animal (e.g., cow, horse, pig) or a pet (e.g., dog, cat). More preferably, the subject is a human. The subject may also be a subject in need of cancer treatment. A subject in need of cancer treatment can be a subject who has been diagnosed as having a disorder characterized by unwanted, rapid cell proliferation. Such disorders include, but are not limited to cancers and precancerous conditions.

Tocopheryl derivatives can be used to both treat and prevent cancer. As used herein, the term “prevention” includes either preventing the onset of a clinically evident unwanted cell proliferation altogether or preventing the onset of a preclinically evident stage of unwanted rapid cell proliferation in individuals at risk. Also intended to be encompassed by this definition is the prevention of metastasis of malignant cells or to arrest or reverse the progression of malignant cells. This includes prophylactic treatment of those at risk of developing precancers and cancers.

Cancer cells contain genetic damage that has resulted in the relatively unrestrained growth of the cells. The genetic damage present in a cancer cell is maintained as a heritable trait in subsequent generations of the cancer cell line. The cancer treated by the method of the invention may be any of the forms of cancer known to those skilled in the art or described herein. Cancer that manifests as both solid tumors and cancer that instead forms non-solid tumors as typically seen in leukemia can be treated. Based on the prevalence of an increase in aerobic glycolysis in all types of cancer, the present invention provide methods for treating a subject that is afflicted with various different types of cancers, including carcinoma, sarcoma, and lymphoma. Examples of types of cancer that can be treated using the compounds of the invention include ovary, colon, lung, breast, thyroid, and prostate cancer, while additional embodiments are directed to only prostate cancer, breast cancer, and pancreatic cancer. A preferred cancer for treatment by tocopheryl derivatives of the present invention is prostate cancer.

The effectiveness of cancer treatment may be measured by evaluating a reduction in tumor load or decrease in tumor growth in a subject in response to the administration of the tocopheryl derivative. The reduction in tumor load may be represent a direct decrease in mass, or it may be measured in terms of tumor growth delay, which is calculated by subtracting the average time for control tumors to grow over to a certain volume from the time required for treated tumors to grow to the same volume.

The compounds of the present invention may be administered alone or in conjunction with other antineoplastic agents or other growth inhibiting agents or other drugs or nutrients, as in an adjunct therapy. The phrase “adjunct therapy” or “combination therapy” in defining use of a compound described herein and one or more other pharmaceutical agents, is intended to embrace administration of each agent in a sequential manner in a regimen that will provide beneficial effects of the drug combination, and is intended as well to embrace co-administration of these agents in a substantially simultaneous manner, such as in a single formulation having a fixed ratio of these active agents, or in multiple, separate formulations for each agent.

For the purposes of combination therapy, there are large numbers of antineoplastic agents available in commercial use, in clinical evaluation and in pre-clinical development, which could be selected for treatment of cancers or other disorders characterized by rapid proliferation of cells by combination drug chemotherapy. Such antineoplastic agents fall into several major categories, namely, antibiotic-type agents, alkylating agents, antimetabolite agents, hormonal agents such as antiandrogens, immunological agents, interferon-type agents and a category of miscellaneous agents. Alternatively, other anti-neoplastic agents, such as metallomatrix proteases inhibitors (MMP), such as MMP-13 inhibitors, or α,β₃ inhibitors may be used. Suitable agents which may be used in combination therapy will be recognized by those of skill in the art. Similarly, when combination with radiotherapy is desired, radioprotective agents known to those of skill in the art may also be used. Treatment using compounds of the present invention can also be combined with treatments such as hormonal therapy, proton therapy, cryosurgery, and high intensity focused ultrasound (HIFU), depending on the clinical scenario and desired outcome.

Candidate agents may be tested in animal models. Typically, the animal model is one for the study of cancer. The study of various cancers in animal models (for instance, mice) is a commonly accepted practice for the study of human cancers. For instance, the nude mouse model, where human tumor cells are injected into the animal, is commonly accepted as a general model useful for the study of a wide variety of cancers (see, for instance, Polin et al., Investig. New Drugs, 15:99-108 (1997)). Results are typically compared between control animals treated with candidate agents and the control littermates that did not receive treatment. Transgenic animal models are also available and are commonly accepted as models for human disease (see, for instance, Greenberg et al., Proc. Natl. Acad. Sci. USA, 92:3439-3443 (1995)). Candidate agents can be used in these animal models to determine if a candidate agent decreases one or more of the symptoms associated with the cancer, including, for instance, cancer metastasis, cancer cell motility, cancer cell invasiveness, or combinations thereof.

In some embodiments, the cancer is a cancer involving Akt signaling dysregulation. Akt, also known as Protein Kinase B (PKB), is a serine/threonine-specific protein kinase that plays a key role in multiple cellular processes such as glucose metabolism, apoptosis, cell proliferation, transcription and cell migration. The inventors have shown that selective Akt dephosphorylation is attributable to a mechanism whereby tocopheryl derivatives facilitate the co-recruitment of Akt and PHLPP1 to the plasma membrane through PH domain recognition. In addition, evidence indicates that PHLPP1 and PTEN form a tumor suppressor network that modulates the P13K-Akt signaling axis, which is often dysregulated in the course of tumorigenesis. Molina et al., Oncogene, 31, 1264-74 (2012) or antiandrogen therapy Carver et al., Cancer Cell, 19, 575-586 (2011).

In another aspect, a method of inactivating Akt in a tumor cell by contacting the tumor cell with an effective amount of a compound according to formula I, as defined herein, is provided. Contacting the tumor cell, as defined herein, refers to placing the compound in an environment where the compound will quickly interact with the tumor cell, such as by delivering the compound to the buffer in a tumor cell culture where the compound will diffuse through the buffer to reach the tumor cells. The method of contacting the tumor cell can be used to contact a tumor cell that is either in vivo or in vitro. When contacting a tumor cell that is in vivo, the compound may be directly administered to the site where tumor cells are present, or the compound may be administered in a formulation that will release the tumor cell in vivo such that a significant quantity of the compound reaches tumor cells that are present in the in vivo environment. As described herein, tocopheryl derivatives facilitate the co-recruitment of Akt and PHLPP1 to the plasma membrane through PH domain recognition, and have a natural affinity for the plasma membrane. Accordingly, in some embodiments, the Akt is inactivated by the compound while present in the membrane of the tumor cell.

### Administration and Formulation of Tocopheryl Derivatives

The present invention provides a method for administering one or more tocopheryl derivatives in a pharmaceutical composition. Examples of pharmaceutical compositions include those for oral, intravenous, intramuscular, subcutaneous, or intraperitoneal administration, or any other route known to those skilled in the art, and generally involve providing the tocopheryl derivative formulated together with a pharmaceutically acceptable carrier either liposomes or nanaparticles.

When preparing the compounds described herein for oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. The active ingredient may also be administered by injection as a composition wherein, for example, saline, dextrose or water may be used as a suitable carrier.

For intravenous, intramuscular, subcutaneous, or intraperitoneal administration, the compound may be combined with a sterile aqueous solution which is preferably isotonic with the blood of the recipient. Such formulations may be prepared by dissolving solid active ingredient in water containing physiologically compatible substances such as sodium chloride, glycine, and the like, and having a buffered pH compatible with physiological conditions to produce an aqueous solution, and rendering said solution sterile. The formulations may be present in unit or multi-dose containers such as sealed ampoules or vials.

Formulations suitable for parenteral administration conveniently comprise a sterile aqueous preparation of the active compound which is preferably made isotonic. Preparations for injections may also be formulated by suspending or emulsifying the compounds in non-aqueous solvent, such as vegetable oil, synthetic aliphatic acid glycerides, esters of higher aliphatic acids or propylene glycol.

The dosage form and amount can be readily established by reference to known treatment or prophylactic regiments. The amount of therapeutically active compound that is administered and the dosage regimen for treating a disease condition with the compounds and/or compositions of this invention depends on a variety of factors, including the age, weight, sex, and medical condition of the subject, the severity of the disease, the route and frequency of administration, and the particular compound employed, the location of the unwanted proliferating cells, as well as the pharmacokinetic properties of the individual treated, and thus may vary widely. The dosage will generally be lower if the compounds are administered locally rather than systemically, and for prevention rather than for treatment. Such treatments may be administered as often as necessary and for the period of time judged necessary by the treating physician. One of skill in the art will appreciate that the dosage regime or therapeutically effective amount of the inhibitor to be administrated may need to be optimized for each individual. The pharmaceutical compositions may contain active ingredient in the range of about 0.1 to 2000 mg, preferably in the range of about 0.5 to 500 mg and most preferably between about 1 and 200 mg. A daily dose of about 0.01 to 100 mg/kg body weight, preferably between about 0.1 and about 50 mg/kg body weight, may be appropriate. The daily dose can be administered in one to four doses per day.

For example, the maximum tolerated dose (MTD) for tocopheryl derivatives can be determined in tumor-free athymic nude mice. Agents are prepared as suspensions in sterile water containing 0.5% methylcellulose (w/v) and 0.1% Tween 80 (v/v) and administered to mice (7 animals/group) by oral gavage at doses of 0, 25, 50, 100 and 200 mg/kg once daily for 14 days. Body weights, measured twice weekly, and direct daily observations of general health and behavior will serve as primary indicators of drug tolerance. MTD is defined as the highest dose that causes no more than 10% weight loss over the 14-day treatment period.

The tocopheryl derivatives can also be provided as pharmaceutically acceptable salts. The phrase “pharmaceutically acceptable salts” connotes salts commonly used to form alkali metal salts and to form addition salts of free acids or free bases. The nature of the salt is not critical, provided that it is pharmaceutically acceptable. Suitable pharmaceutically acceptable acid addition salts of compounds of formula I may be prepared from an inorganic acid or from an organic acid. Examples of such inorganic acids are hydrochloric, hydrobromic, hydroiodic, nitric, carbonic, sulfuric, and phosphoric acid. Appropriate organic acids may be selected from aliphatic, cycloaliphatic, aromatic, araliphatic, heterocyclic, carboxylic, and sulfonic classes of organic acids, examples of which include formic, acetic, propionic, succinic, glycolic, gluconic, lactic, malic, tartaric, citric, ascorbic, glucoronic, maleic, fumaric, pyruvic, aspartic, glutamic, benzoic, anthranilic, mesylic, salicylic, p-hydroxybenzoic, phenylacetic, mandelic, ambonic, pamoic, methanesulfonic, ethanesulfonic, benzenesulfonic, pantothenic, 2-hydroxyethanesulfonic, toluenesulfonic, sulfanilic, cyclohexylaminosulfonic, stearic, algenic, γ-hydroxybutyric, galactaric, and galacturonic acids. Suitable pharmaceutically acceptable base addition salts of the compounds described herein include metallic salts made from aluminum, calcium, lithium, magnesium, potassium, sodium, and zinc. Alternatively, organic salts made from N,N′-dibenzylethylenediamine, chloroprocaine, choline, diethanolamine, ethylenediamine, meglumine (N-methylglucamine) and procaine may be used form base addition salts of the compounds described herein. All of these salts may be prepared by conventional means from the corresponding compounds described herein by reacting, for example, the appropriate acid or base with the compound.

### Preparation of Tocopheryl Derivatives

Compounds of the invention may be synthesized by synthetic routes that include processes analogous to those well known in the chemical arts, particularly in light of the description contained herein. In particular, the preparation of a number of tocopheryl derivatives is described in Example 2. The starting materials are generally available from commercial sources such as Aldrich Chemicals (Milwaukee, Wis., USA) or are readily prepared using methods well known to those skilled in the art (e.g., prepared by methods generally described in Louis F. Fieser and Mary Fieser, Reagents for Organic Synthesis, v. 1-19, Wiley, New York, (1967-1999 ed.) and similar texts known to those skilled in the art.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Vitamin E Facilitates PHLPP-Mediated Akt Inactivation

α- and γ-tocopherol can mediate the site-specific dephosphorylation of the kinase Akt at Ser⁴⁷³ with activities paralleling their respective antiproliferative potencies in prostate cancer cells. Moreover, this selective Akt dephosphorylation is attributable to a mechanism whereby α- and γ-tocopherol facilitate the co-recruitment of Akt and pleckstrin homology (PH) domain leucine-rich repeat protein phosphatase (PHLPP), a Ser⁴⁷³-specific Akt protein phosphatase, to the plasma membrane through PH domain recognition. This tocopherol-induced activation of PHLPP is noteworthy in light of the tumor suppressor role of PHLPP1 in prostate cancer by counteracting the functional loss of phosphatase and tensin homolog (PTEN) in suppressing Akt activation. Chen et al., Cancer Cell 20, 173 (2011). Moreover, structural modification of these tocopherols enhanced this activity, thereby providing a rationale for optimizing tocopherols to generate a series of potent Akt pathway-targeted agents.

### Materials and Methods

### Cell Lines, Culture, Reagents and Antibodies.

The prostate cancer cell lines, LNCaP and PC-3, were purchased from American Type Culture Collection (Manassas, Va.) and maintained in RPMI 1640 medium containing 10% heat-inactivated fetal bovine serum at 37° C. in a humidified incubator containing 5% CO₂. The LNCaP-abl cells were a generous gift from Dr. Qianben Wang (The Ohio State University). Normal prostate epithelial cells were purchased from Lonza (Walkersville, Md.) and were cultured in the vendor-recommended defined prostate epithelial cell growth medium. For experiments, LNCaP cells were plated on poly-D-lysine-coated culture flasks at a density of 12,000 cells per cm² surface area for 24 hours, followed by treatment with test agents in serum-free RPMI 1640 medium. The α- and γ-tocopherols were purchased from Sigma-Aldrich (St. Louis, Mo.). The tocopherol derivatives, α- and γ-VE5, were synthesized by the inventors.

[³²P]Orthophosphate was purchased from PerkinElmer Life Sciences (Waltham, Mass.). Di-octanoyl PIP3 was obtained from CellSignals, Inc. (Columbus, Ohio). LY-294002 was purchased from LC Laboratories (Woburn, Mass.). Antibodies specific to Akt phosphorylated at Ser⁴⁷³, Akt phosphorylated at Thr³⁰⁸, Akt, PDK-1 and β-actin were purchased from Cell Signaling Technology, Inc. (Beverly, Mass.), PHLPP from Novus Biologicals (Littleton, Colo.), Na+-K+ ATPase and ILK from Santa Cruz Biotechnology, Inc. (Santa Cruz, Calif.), flotillin-2 from BD Biosciences (Franklin Lakes, N.J.), and BTK from BD Biosciences-Pharmingen (San Diego, Calif.). Alexa Fluor 555- and 488-conjugated goat anti-rabbit and anti-mouse IgG were purchased from Invitrogen (Carlsbad, Calif.), and anti-mouse and anti-rabbit secondary antibodies were obtained from Jackson ImmunoResearch Laboratories (West Grove, Pa.).

### Viability Assay.

LNCaP cells were plated into poly-D-lysine-coated 96-well plates and PC-3 and LNCaP-abl cells into uncoated plates at the density of 5,000 cells per well in the presence of 10% FBS. Normal prostate epithelial cells were plated into uncoated plates at a density of 8,000 cells per well. Exposure to test agents in serum-free medium was initiated 24 hours later. After 24 hours of treatment, cells were incubated with MTT [3-(4,5-dimethylthiazol-2-yl)-2,5-diphenyl-2H-tetrazolium bromide] (TCI America; 0.5 mg/ml, final concentration) for an additional 2 hours. The medium was then removed from each well and replaced with DMSO to dissolve the reduced MTT dye for subsequent colorimetric measurement of absorbance at 595 nm. Cell viabilities are calculated as percentages of that in the corresponding vehicle-treated control group.

### Cell Lysis and Immunoblotting.

Cells were exposed to the test agents in 10-cm dishes for 24 hours and then collected by scraping. The cell pellets were washed once with PBS, and then lysed at 4° C. over 30 min of incubation in a sodium dodecyl sulfate (SDS) lysis buffer containing 1% SDS, 50 mM Tris HCl (pH 8.1), 10 mM EDTA and SIGMAFAST protease inhibitor cocktail (Sigma-Aldrich). The cell debris was pelleted by centrifugation for 20 min at 14,000×g, and 1 μl of each supernatant was used for determination of protein concentration using a colorimetric bicinchoninic assay (Pierce, Rockford, Ill.). The remaining sample was added to equal volume of 2×SDS-polyacrylamide gel electrophoresis sample loading buffer (62.5 mM Tris-HCl, pH 6.8, 4% SDS, 5% β-mercaptoethanol, 20% glycerol, 0.1% bromophenol blue), followed by incubation in boiling water for 5 min. Equivalent amounts of total protein were resolved in SDS-polyacrylamide gels, and then transferred to nitrocellulose membranes using a semidry transfer cell. The transblotted membrane was washed twice with Tris-buffered saline containing 0.1% Tween 20 (TBST). After blocking with TBST containing 5% nonfat milk for 40 min, the membrane was incubated with the appropriate primary antibody (1:1000) in TBST-1% nonfat milk at 4° C. overnight. The membrane was then washed three times with TBST for a total of 15 min, followed by incubation with goat anti-rabbit or anti-mouse IgG-horseradish peroxidase conjugates (1:2000) for 1 hour at room temperature and four washes with TBST for a total of 1 hour. The immunoblots were visualized by enhanced chemiluminescence (GE Healthcare Life Sciences).

### Immunocytochemistry and Confocal Microscopy

After 24 hours treatment, LNCaP cells were fixed in 4% formaldehyde for 20 min before permeabilization with 0.1% Triton X-100 in PBS at room temperature for 1 hour followed by incubation in 1% FBS (in PBS) for 1 hour. Cells were stained for endogenous PH domain-containing proteins (Akt, PHLPP, PDK-1, ILK, BTK) by incubation with specific antibodies (1:100), followed by incubation with the Alexa Fluor 555-conjugated goat anti-rabbit or Alexa Fluor 488-conjugated goat anti-mouse IgG (1:200) at room temperature for 2 hours. Both primary and secondary antibodies were diluted in incubation buffer containing 0.1% Triton X-100 and 0.2% bovine serum albumin in PBS. The cells were washed in PBS after each step and mounted using VECTASHIELD mounting medium supplemented with DAPI (Vector Laboratories Inc., Burlingame, Calif.). The slides were allowed to set for at least 4 hours before confocal images were acquired using a Zeiss LSM 510 inverted confocal laser scanning microscope operated with Zeiss LSM 510 software. Image analysis was performed using ImageJ (NIH) software.

### Co-Immunoprecipitation of Akt-PHLPP Complexes.

The cytosolic fractions of treated LNCaP cells were isolated as described previously with minor modification. Adam et al., Cancer Res 67, 6238 (2007). Briefly, cells were resuspended in cytosol buffer (50 mM HEPES, pH7.4, 10 mM NaCl, 1 mM MgCl₂, 0.5 M EDTA, 1 mM phenylmethylsulfonyl fluoride and 1 mM Na₃VO₄) and then disrupted by 10 passages through a 26 G needle. After centrifugation of the cell homogenates at 14,000×g for 20 mM at 4° C., the supernatants were collected as the cytosolic fractions. Triton-soluble fractions were extracted from the membrane pellets by resuspension in Triton X-100-containing lysis buffer (50 mM Tris-HCl, pH 7.5, 150 mM NaCl, 1 mM EGTA, 10 mM MgCl₂, 0.5% Triton X-100, and protease inhibitor mixture) and disruption by 10 passages through a 26 G needle. After centrifugation of the homogenates at 14,000×g for 20 min at 4° C., the supernatants were collected as the Triton-soluble membrane fractions. For co-immunoprecipitation, the Triton-soluble samples were incubated with 30 μl protein A/G agarose beads for 30 min at 4° C. to eliminate nonspecific binding, after which the supernatants were collected by centrifugation at 6,000×g for 3 min. The samples were then incubated with 10 μl of anti-Akt-bound agarose beads overnight at 4° C. Non-specific IgG was used as a negative control. After brief centrifugation, the beads were collected and washed once with Triton X-100-containing lysis buffer, once with wash buffer 1 (50 mM Tris, pH 7.5, 500 mM NaCl, and 0.2% Triton X-100), and once with wash buffer 2 (10 mM Tris, pH 7.5, and 0.2% Triton X-100). Immunoprecipitates were then eluted from the beads by the addition of 70 μl 2× Laemmli sample buffer followed by boiling at 95° C. for 5 min, and subjected to Western blot analysis.

### Isolation of Lipid Raft-Containing Membrane Subfractions

Triton-insoluble membrane constituents were isolated from 5×10⁶ treated LNCaP by detergent extraction as described previously. Zhuang et al., J Clin Invest 115, 959 (2005). Lipid raft-containing membrane subfractions were isolated by centrifugation through sucrose density gradients as described previously with the following minor modifications. Royer et al., J Biol Chem 284, 15826 (2009). After treatment for 24 h in serum-free medium, 5×10⁶ LNCaP cells per treatment group were collected, washed and then disrupted on ice by passage through a 26G needle 6 times. After centrifugation through a sucrose gradient, consecutive 1.2-ml fractions were collected from the top of the gradient and stored at −20° C. until western blot analysis was performed to assess the abundance of phosphorylated Ser473 in Akt, total Akt, PHLPP1, and flotillin-2 in each fraction.

### Transfections and Ectopic Expression of CA-Akt

Transient transfections were performed using the Amaxa® Nucleofector system or Lipofectamine™ 2000 (Invitrogen) according to the manufacturer's protocol. Nucleofection utilized a commercially available Nucleofector kit (KitR-program T-09) (Lonza, Inc., Walkersville, Md.). LNCaP cells were transfected with the plasmid encoding hemagglutinin-tagged CA-Akt (pcDNA-HA-PKB-T308D-S473D) obtained from Addgene (Cambridge, Mass.). For siRNA-mediated knockdown of PHLPP, LNCaP cells were transfected with specific siRNA or scrambled siRNA as a control according to manufacturer's instructions (Dharmacon). The expression of the cloned protein and the knockdown of PHLPP were confirmed by immunoblotting.

### Flow Cytometry

For assessment of apoptosis, treated LNCaP cells were stained with annexin V-FITC and propidium iodide (Invitrogen) according to the manufacturer's protocol. Unstained vehicle-treated control cells, control cells stained with Annexin V-FITC only, and control cells stained with PI only were used for gating and background subtraction. For each sample, 10,000 cells were acquired for flow cytometry using a FACSCalibur cytometer (BD Biosciences, San Jose, Calif.). Data were analyzed using the FloJo software program.

### Autoradiographic Determination of Phosphoinositide Formation

Evaluation of phosphoinositide formation in tocopherol-treated LNCaP cells was performed as described previously with the following modification. Chen et al., J Biol Chem 280, 38879 (2005). After labeling cells with [³²P]orthophosphate (HCl-free) in phosphate- and serum-free Dulbecco's modified Eagle's medium, cells were exposed to α-tocopherol for 6 hours in serum-free RPMI 1640 medium.

### Cholesterol Measurement

Three hundred μl aliquots of each of the fractions collected from the sucrose gradient preparation of membrane subfractions were used for measurement of cholesterol concentrations. Each sample was extracted with 300 μl of a chloroform:isopropanol:NP-40 mixture (7:11:0.1). The organic phase was collected by centrifugation at 15,000×g for 10 min and then air-dried at 50° C. to remove chloroform. Trace amounts of organic solvent were removed by evaporation. The cholesterol content in each sample was analyzed using a Total Cholesterol Assay Kit (Cell Biolabs, San Diego, Calif.).

### In Vivo Study

Male athymic nude mice (Hsd:Athymic Nude-Foxn1^{nu/nu}, 5-7 weeks of age) were purchased from Harlan Laboratories and group-housed under conditions of constant photoperiod (12 hours light: 12 hours dark) with ad libitum access to sterilized food and water. Ectopic tumors were established in athymic nude mice by subcutaneous injection of 1×10⁶ PC-3-luc or 2×10⁶ LNCaP-abl cells in a total volume of 0.1 mL serum-free medium containing 50% Matrigel (BD Biosciences). The establishment and growth of tumors were monitored weekly by measurement with calipers (tumor volume=width²×length×0.52) and bioluminescence (PC-3-luc) using the IVIS™ imaging system (Xenogen Corporation, Alameda, Calif.). For bioluminescent imaging, mice anesthetized with isoflurane were imaged at 15, 20 and 25 min after intraperitoneal administration of firefly luciferin (150 mg/kg; Caliper Life Sciences) to capture maximal luminescence. Data acquisition and analysis were achieved using the Living Image® software (Xenogen). Mice with established tumors (mean starting tumor volume ±SE: PC-3-luc, 75.3±4.5 mm³; LNCaP-abl, 119.6±7.5 mm³) were randomized to two groups (n=7-8) that received daily intraperitoneal injections of γ-VE5 at 50 mg/kg body weight or vehicle (physiological saline/polyethylene glycol 400/DMSO/Tween 80; 65:20:10:5 by volume) for 21 days. Body weights and tumor burdens were measured weekly. At the study endpoint, mice were euthanized, and blood was collected by cardiac puncture from all mice and submitted to The Ohio State University Comparative Pathology and Mouse Phenotyping Shared Resource for determination of complete blood counts and serum chemistry.

### Construction of Plasmids Expressing GFP-Tagged Akt, PH Domain of Akt and PH Domain-Deleted (ΔPH)-Akt

PC-3 cell cDNA was used as template for the PCR amplification of sequences for the full length wild-type Akt and PHLPP, and for the PH domains of Akt and PHLPP. PCR products were inserted into the pcDNA3.1/CT-GFP-TOPO vector (Invitrogen). To create the GFP-ΔPH plasmids, the GFP-full length Akt and PHLPP-expressing plasmids were amplified using primers that omitted the PH-domain-coding region of Akt or PHLPP in the product.

### Construction of Plasmids and Expression of GST-PH Domain Fusion Proteins

The cDNA fragments corresponding to the putative PH domain sequences of PHLPP1 (+115 to +372), PDK1 (+1225 to +1671), Akt1 (+1 to +447) and ILK (+538 to +646) were PCR-amplified using the plasmids pcDNA-HA-PHLPP1, pWZL-Neo-Myr-FLAG-PDPK1, pcDNA-HA-PKB-T308D-S473D, and pCMV-SPORT-ILK (Addgene) as templates. The PCR products were then cloned into EcoRI/XhoI sites of the pGEX-4T1 expression vector (GE Healthcare Life Sciences) to generate four constructs (pGEX-4T1-PHLPP1, pGEX-4T1-PDPK1, pGEX-4T1-AKT1, pGEX-4T1-ILK) for the expression of GST-PH domain fusion proteins. The mutated Akt1 Y38G PH domain was generated from pGEX-4T1-AKT1 by site-directed mutagenesis using the QuikChange II XL Site-Directed Mutagenesis Kit (Agilent Technologies). The expression and purification of GST-PH domain fusion proteins were performed essentially as described previously. Thomas et al., Curr Biol 12, 1256 (2002). The purified PH domains were analyzed by SDS-PAGE using an overloaded gel with purities were estimated to be >95% using ImageJ software.

### Molecular Modeling

The primary sequence of human Akt (National Center for Biotechnology Information, NP_{—}001014432.1) and the crystal structure of the human Akt-PH domain (RCSB Protein Data Bank [PDB], 2UZS) were used for the molecular docking simulations. The structures of α/γ-VE5 were constructed by geometry-optimization with CHARMM force field calculation. Docking of α- or γ-VE5 into the Akt-PH domain was performed using the CHARMM-based molecular docking algorithm implemented in the Discovery Studio 2.1 program (Accelrys, Inc., San Diego, Calif.). The flexibility of the compounds was accounted for by including different orientations and rotatable torsion angles in the docking procedure. Accordingly, 10⁸ conformation structures were generated, among which representatives of 10² stable conformations were analyzed. Homologies among the PH domain sequences of human Akt, PHLPP1, PDK1, BTK, and ILK, (NP_{—}001014432.1, NP_{—}919431.2, NP_{—}002604.1, NP_{—}000052.1 and NP_{—}001014795.1, respectively) were identified by sequence alignment using the FASTA program. The corresponding PH domain structures were retrieved from the Research Collaboratory for Structural Bioinformatics (RCSB) Protein Data Bank (PDB) (3A8N, 1W1G, 1B55, and 3REP, respectively). The main secondary structural elements of each protein were defined from the PDB coordinates using the DSSP program. W. Kabsch, C. Sander, Biopolymers 22, 2577 (1983).

### Surface Plasmon Resonance (SPR) Spectroscopy

Binding experiments were performed using a Biacore T100 system (GE Healthcare, Piscataway, N.J.). The GST-PH domain fusion proteins were immobilized on a CM5 S Sensorchip using Biacore's Amine Coupling Kit to a level of 17,000 response units. VE5 compounds and di-octanoyl PIP₃ at concentrations ranging from 0.1 to 100 μM and 1 to 20 μm, respectively, were injected at a high flow rate (30 μL/min) over the biosensor surface for binding analyses. DMSO concentrations in all samples and running buffer were 1% (v/v) or less. Data were analyzed using Biacore T100 Evaluation Software.

### Statistical Analysis

In vitro experiments were performed using 3 to 6 biological replicates per group in at least three independent experiments. Data were assessed for normality using the Shapiro-Wilk test. For normally distributed data, differences between group means were analyzed for statistical significance using the Student's t-test or ANOVA followed by Dunnett's or Games-Howell's post-hoc test for multiple comparisons. For data that failed to meet the assumption of normality, group means were compared using the Mann-Whitney U test or the Kruskal-Wallis H test. Differences were considered significant at P<0.05. Analyses were performed using SPSS Statistics (version 20, IBM Corp.).

### Results

### α- and γ-Tocopherols Cause the Site-Specific Dephosphorylation of Akt at Ser-473

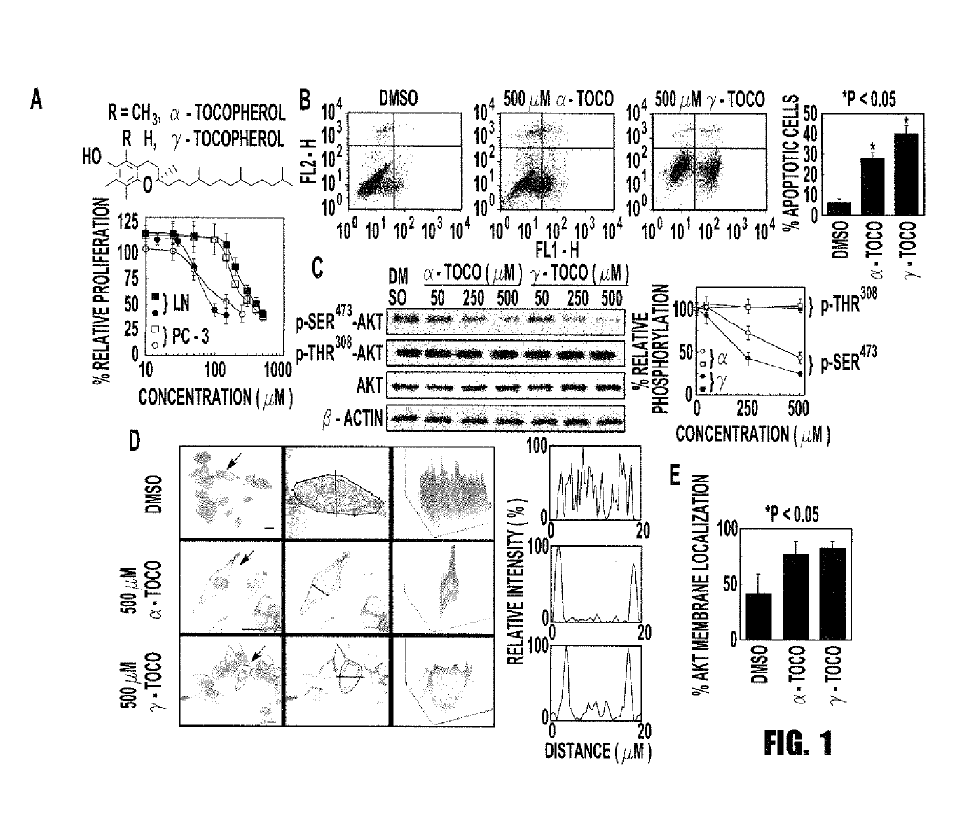

The antiproliferative activities of α- and γ-tocopherol were examined in two prostate cancer cell lines, LNCaP (androgen-responsive) and PC-3 (androgen-independent), both of which exhibit activated Akt resulting from loss of PTEN function. Both cell lines were equally susceptible to the antiproliferative effect of these compounds, and γ-tocopherol (IC₅₀˜100-150 μM) was more potent than α-tocopherol (IC₅₀˜400 μM) (FIG. 1A). This cytotoxic effect was attributable to apoptosis (FIG. 1B) and was cancer cell-specific, because normal prostate epithelial cells were resistant to apoptosis induced by either α- or γ-tocopherol. Because vitamin E inhibits cancer cell proliferation by targeting Akt (Lee et al., Clin Cancer Res 15, 4242 (2009)), the effects of α- and γ-tocopherol on the phosphorylation of Akt were examined in both cell lines. Both tocopherols dose-dependently reduced the phosphorylation at Ser⁴⁷³ in Akt, with relative potencies paralleling those of growth inhibition, without altering phosphorylation at Thr³⁰⁸ in both LNCaP (FIG. 1C) and PC-3 cells.

### Tocopherols Promote the Dephosphorylation of Ser⁴⁷³ in Akt by Recruiting Akt and PHLPP to the Plasma Membrane

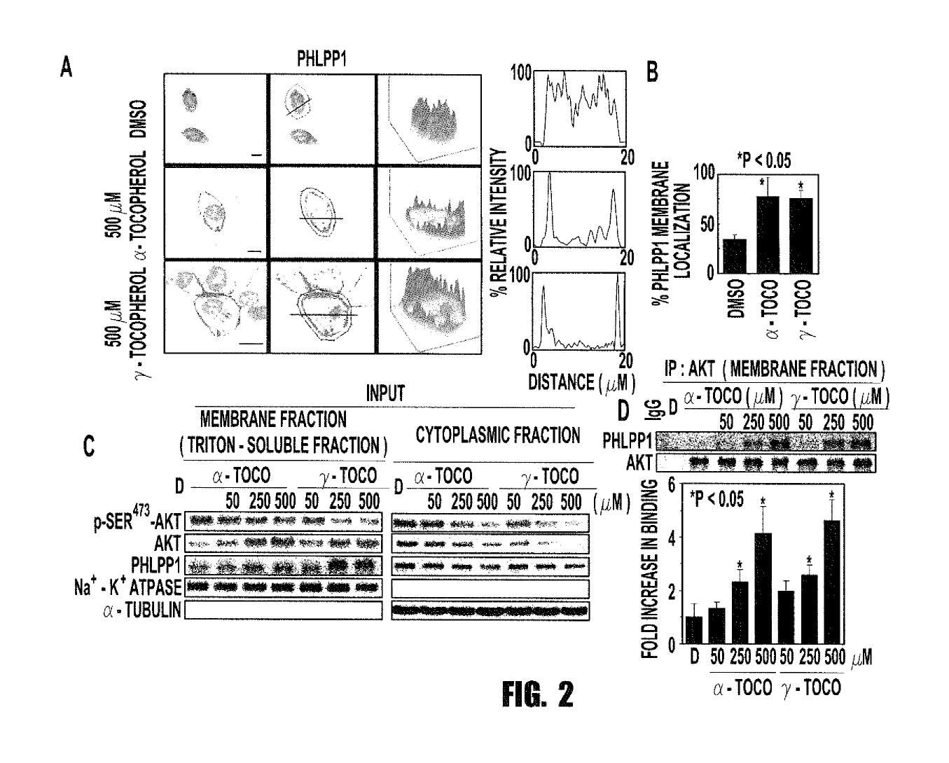

These findings suggest the antitumor action for the tocopherols occurs through the site-specific dephosphorylation and consequent inactivation of Akt. Because tocopherols are localized within the membrane after uptake by cells, the inventors postulated that this tocopherol-facilitated dephosphorylation of Ser⁴⁷³ in Akt would be a membrane-associated event. In support of this premise, immunocytochemical analysis showed that Akt localized to the membrane in response to α- and γ-tocopherol in LNCaP cells (FIGS. 1D and E). This tocopherol-mediated membrane localization of Akt is similar to the reported PIP₃-facilitated recruitment of Akt through PH domain recognition. L. C. Cantley, Science 296, 1655 (2002); I. Vivanco, C. L. Sawyers, Nat Rev Cancer 2, 489 (2002). Thus, radiometric analysis of phosphoinositide production in tocopherol-treated LNCaP cells was performed, which showed that PIP₃ concentrations were unaffected by exposure to 500 μM α-tocopherol (FIG. 2A). In addition, pre-treatment of LNCaP cells with the phosphoinositide 3-kinase (PI3K) inhibitor LY-294002 did not affect the ability of α- and γ-tocopherol to facilitate the membrane recruitment of Akt (FIG. S2A). Together, these findings suggest that phosphatidylinositol 3,4,5-trisphosphate (PIP₃) is not required for tocopherol-induced Akt recruitment. Moreover, because the rictor-mTOR (mammalian target of rapamycin) complex (mTORC2) facilitates phosphorylation of Akt at Ser⁴⁷³ in many types of cells, the concentration-dependent effects of α- and γ-tocopherol on the abundance of mTOR and rictor were examined, and the phosphorylation of the downstream targets of mTORC2, including serum- and glucocorticoid-inducible kinase (SGK) and protein kinase C (PKC)α. The data show that α- and γ-tocopherol had no effect on the phosphorylation of SGK or PKCα, suggesting that mTORC2 is not involved in tocopherol-induced dephosphorylation of Akt.

Compared to Akt activation, the mechanisms by which Akt is inactivated are less well defined. Bayascas et al., Mol Cell 18, 143 (2005). Protein phosphatase 2A and PHLPP facilitate the dephosphorylation of Akt at Thr³⁰⁸ and Ser⁴⁷³, respectively. Because PHLPP, like Akt, also contains a PH domain, the inventors hypothesized that α- and γ-tocopherol induced the recruitment of both Akt and PHLPP to the plasma membrane through their respective PH domains, leading to their co-localization and subsequent dephosphorylation of Ser⁴⁷³ in Akt. Immunocytochemical analysis showed that α- and γ-tocopherol facilitated the membrane localization of PHLPP1 (FIGS. 2B and C) in a manner similar to that of Akt in LNCaP cells. It has been reported that the PHLPP family consists of two isoforms, PHLPP1 and PHLPP2, which dephosphorylate distinct isoforms of Akt. As PHLPP1 and PHLPP2 exhibit similar domain structures (Brognard et al., Mol Cell 25, 917 (2007)), the ability of α- and γ-tocopherol to facilitate the membrane translocation of these two isoforms was examined. Western blot analysis of the membrane fraction of treated cells confirmed that α- and γ-tocopherol increased the membrane localization of Akt in concert with both isoforms of PHLPP in a dose-dependent manner, which was accompanied by parallel decreases in phosphorylation of Ser⁴⁷³ in Akt and the abundance of cytoplasmic Akt and PHLPP1 (FIG. 2D, and FIG. S2D). However, no apparent changes in the cytoplasmic PHLPP2 were noted, which might be reflective of the relative abundances of PHLPP1 versus PHLPP2 in the cytoplasm. Despite this discrepancy, co-immunoprecipitation analysis of Akt-PHLPP complexes from the membrane fraction revealed dose-dependent increases in the association of Akt with both PHLPP1 and PHLPP2 in response to α- and γ-tocopherol (FIG. 2E), indicating that tocopherols recognized both isoforms for binding.

From a mechanistic perspective, these findings contrast with the general notion that Akt membrane translocation is integral to its activation by phosphorylation at Thr³⁰⁸ and Ser⁴⁷³ by phosphoinositide-dependent protein kinase 1 (PDK-1) and mTORC2, respectively.

Truncation of the aliphatic side chain enhances the ability of α- and γ-tocopherol to induce apoptotic death through inactivation of Akt and membrane recruitment of Akt and PHLPP.

Based on the above findings, the inventors proposed that the ability of α- and γ-tocopherol to recruit Akt and PHLPP to the plasma membrane was mediated through interactions of the chroman ring in the polar head group with these proteins at the membrane-cytoplasm interface. Specifically, a thermodynamic interplay between the long aliphatic side chain of α- or γ-tocopherol and the lipid-bilayer membrane was proposed, which might restrict the accessibility of the chroman ring to the cytoplasmic milieu by pulling the head group inward into the membrane. Thus, it was hypothesized that shortening the side chain of the tocopherols would increase the cytoplasmic exposure of the respective polar head groups, thereby enhancing their ability to recruit Akt and PHLPP and thus increasing the tocopherols' antitumor activities.

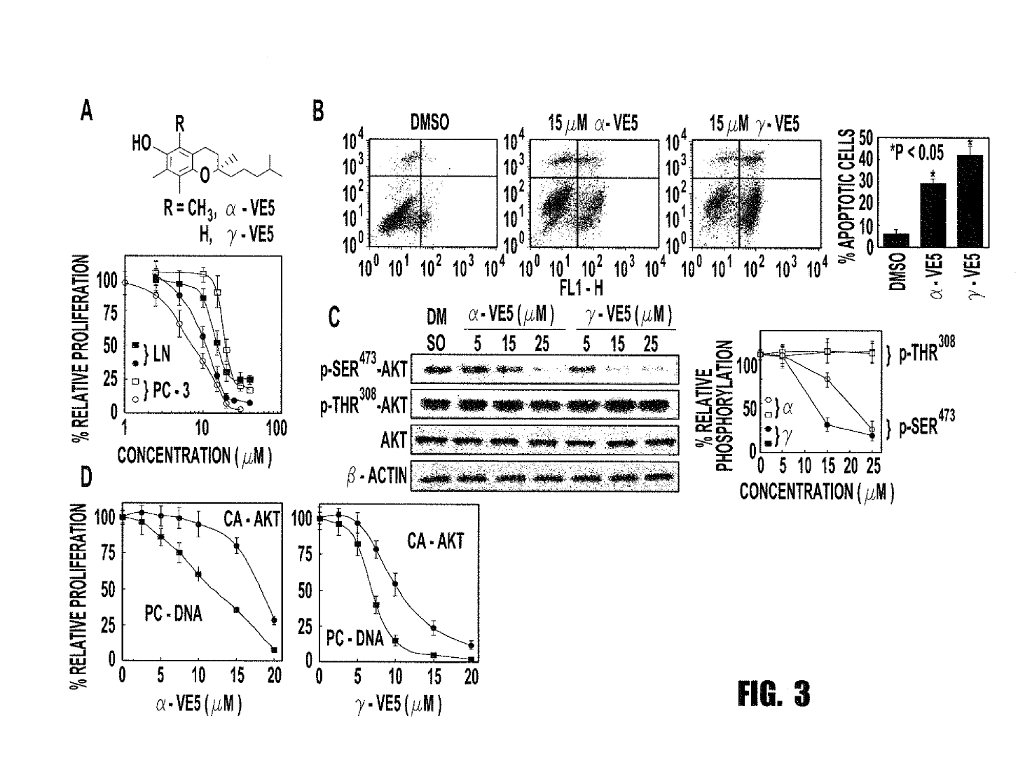

The proof-of-concept of this hypothesis was provided by α- and γ-VE5, which were derived from α- and γ-tocopherol, respectively, by removing two isopranyl units from the respective aliphatic side chains (FIG. 3A, upper panel). α- and γ-VE5 were an order of magnitude more potent than their parent molecules in suppressing the viability of LNCaP and PC-3 cells (IC₅₀ of α-VE5, 15-20 μM; IC₅₀ of γ-VE5, 7-10 μM) (FIG. 3A, lower panel) through apoptosis (FIG. 3B), accompanied by parallel reduction in the phosphorylation of Akt at Ser⁴⁷³ without disturbing that of Thr³⁰⁸ (LNCaP, FIG. 3C). The involvement of this Akt inactivation in α- and γ-VE5-induced cell death was validated by the protective effect of ectopic expression of constitutively active Akt (Akt T308D and S473D; T308D/S473D) (FIG. 3D). As with α- and γ-tocopherol, α- and γ-VE5-mediated inhibition of phosphorylation of Ser⁴⁷³ in Akt was not associated with inhibition of mTORC2 as shown by the lack of changes in the abundance or phosphorylation of mTOR, rictor, SGK, and PKCα in drug-treated cells. Moreover, normal prostate epithelial cells were more resistant to α- and γ-VE5 than malignant cells with IC₅₀ values of approximately 50 and 40 μM, respectively, which represent a 3- to 5-fold difference in potency relative to LNCaP and PC-3 cells.

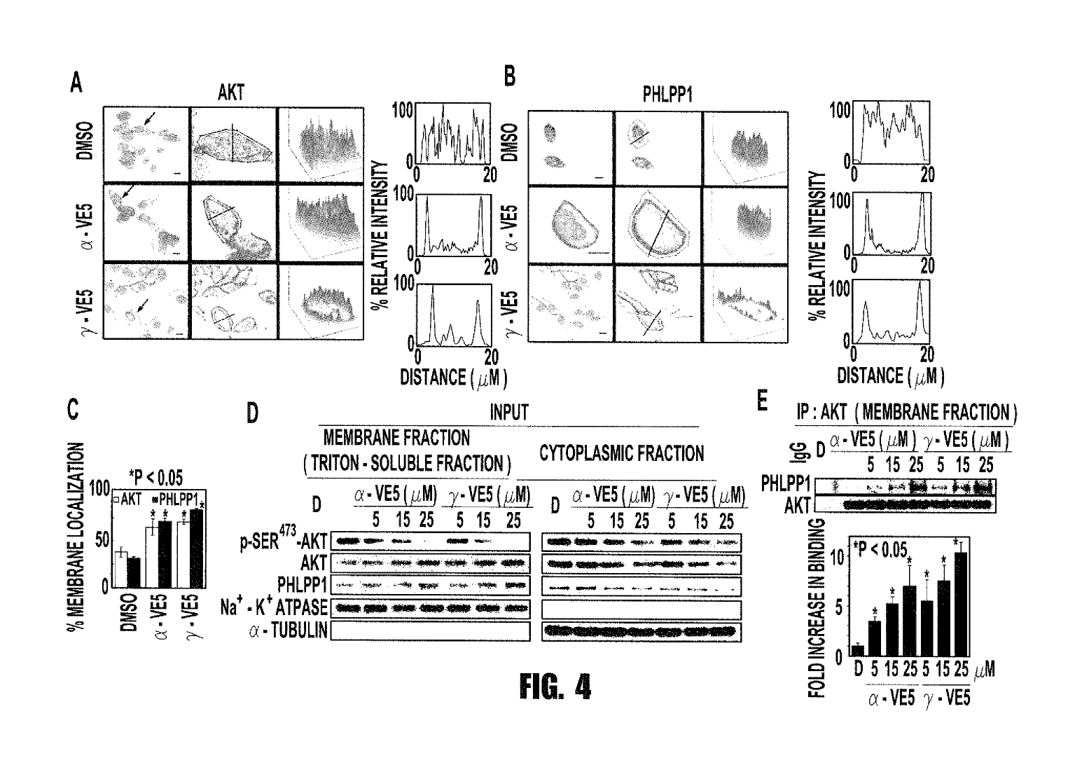

Furthermore, immunocytochemical analysis revealed that α- and γ-VE5 retained the ability of the tocopherols (FIGS. 1D and 2B) to facilitate the membrane recruitment of Akt and PHLPP1 (FIGS. 4A and B, respectively; bar graph, FIG. 4C). This immunocytochemical finding was confirmed by Western blot analysis which showed that exposure of LNCaP cells to α- or γ-VE5 led to concentration-dependent increases in membrane-associated Akt and both isoforms of PHLPP and parallel decreases in the cytoplasmic abundance of Akt and PHLPP1 as well as a decrease in phosphorylation of Ser⁴⁷³ in Akt (FIG. 4D). In addition, co-immunoprecipitation analysis demonstrated concentration-dependent increases in the complex formation of Akt with PHLPP1 and PHLPP2 in (- and γ-VE5-treated cells, further substantiating the role of PHLPP in facilitating drug-induced dephosphorylation of Ser⁴⁷³ in Akt (FIG. 4E).

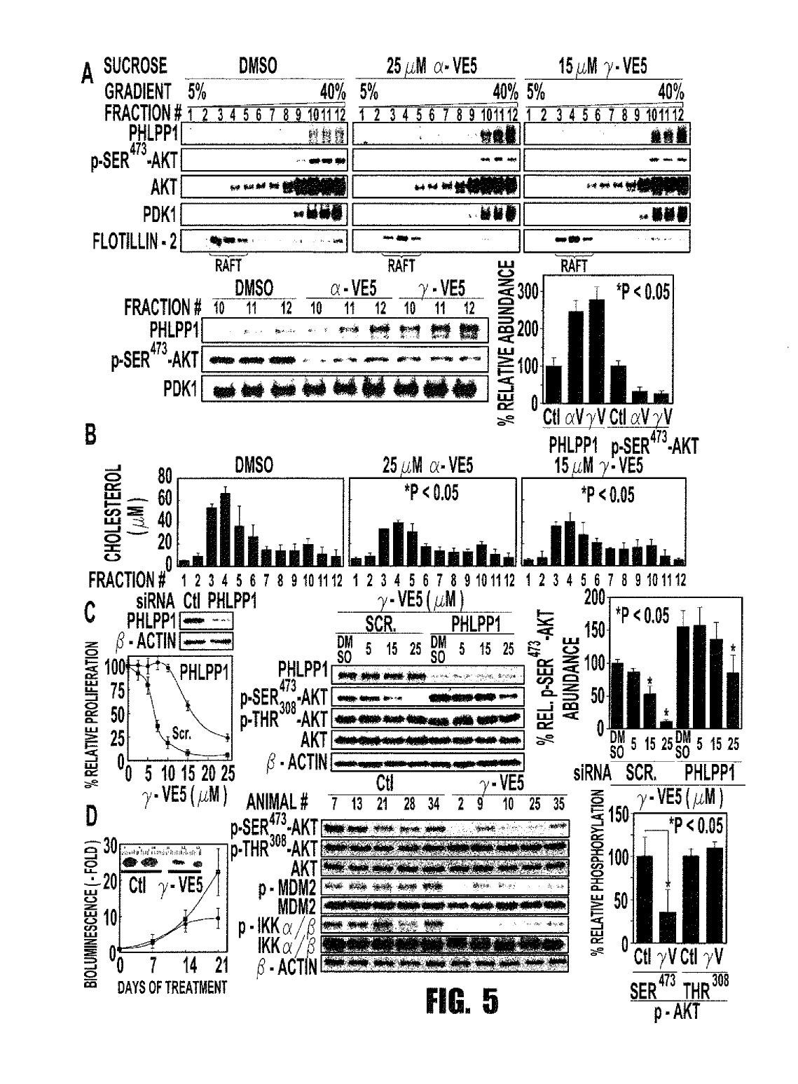

PIP₃-induced membrane recruitment and activation of Akt is mediated through cholesterol-rich lipid rafts of the plasma membrane. Zhuang et al., J Clin Invest 115, 959 (2005). Data on the distribution of α-tocopherol within the cell membrane suggests a non-random distribution, with some studies indicating its association with lipid rafts (Gao et al., Proc Natl Acad Sci USA 108, 14509 (2011)), and others suggesting that it is localized to polyunsaturated fatty acid-rich non-raft domains. Atkinson et al., Mol Nutr Food Res 54, 641 (2010). Consequently, we investigated whether Akt and PHLPP localized to the lipid raft or non-raft microdomain of cell membranes in response to α- and γ-VE5 using density gradient centrifugation. As indicated by the presence of the raft-associated marker flotillin-2, lipid rafts were associated with the low-density fractions, whereas, reminiscent of another report (Adam et al., Cancer Res 67, 6238 (2007)), the majority of Akt and all detectable Akt phosphorylated at Ser⁴⁷³ were present in higher density fractions that corresponded to the non-raft membrane in vehicle-treated cells (FIG. 5A). Moreover, only a small amount of PHLPP1 was found in these Akt-containing, non-raft membrane fractions in vehicle-treated cells. However, after exposure to α- and γ-VE5, the association of PHLPP1 with the membrane increased more than 2.5-fold in these non-raft fractions and was accompanied by a parallel decrease by more than 65% in the phosphorylation of Ser⁴⁷³ in Akt (FIG. 5A). This finding is consistent with the hypothesis that α-tocopherol is localized to polyunsaturated fatty acid-rich non-raft domains.

In light of our finding that α- and γ-VE5 facilitated Aid recruitment preferentially to the non-raft domains without observable increases in Akt binding to raft domains, and evidence suggesting that cholesterol in raft microdomains plays a critical role in facilitating Akt membrane recruitment and activation, we examined the effect of α- and γ-VE5 on the cholesterol content of individual membrane fractions. Both α- and γ-VE5 reduced the cholesterol content in the raft domains (fractions 3 and 4) by as much as 30% (FIG. 5B), suggesting that this VE5-induced reduction in cholesterol content might hamper the PIP₃-mediated recruitment of Akt to membrane raft domains.

To further corroborate the role of PHLPP in mediating tocopherol-induced Akt inactivation, we examined the effect of siRNA-mediated knockdown of PHLPP1 on α- and γ-VE5-induced cell death and the dephosphorylation of Ser⁴⁷³ in Akt in LNCaP cells. Silencing of PHLPP1 expression partially protected cells against the inhibitory effects of γ-VE5 on cell viability and phosphorylation of Akt at Ser⁴⁷³ (FIG. 5C). Similar findings were obtained for α-VE5-treated cells.

### γ-VE5 Suppresses PC-3 and LNCaP-abl Xenograft Tumor Growth In Vivo

The effects of γ-VE5 on tumor growth in vivo were assessed in athymic nude mice bearing subcutaneous xenograft tumors generated from luciferase-expressing PC-3 (PC-3-luc) and LNCaP-abl (a castration-resistant LNCaP subline) cells. LNCaP-abl cells and the parental LNCaP cells were comparably susceptible to the antiproliferative effects of α- and γ-VE5 (IC₅₀, 10 and 7 μM, respectively; FIG. S5D). Mice bearing tumors established from PC-3-luc cells (mean tumor volume ±SE, 75.3±4.5 mm³) or LNCaP-abl cells (mean tumor volume ±SE, 119.6±7.5 mm³) were injected with γ-VE5 or vehicle (n=7-8). Measurements of bioluminescence revealed that treatment with γ-VE5 inhibited PC-3-luc tumor growth relative to vehicle-treated controls at 21 days (P<0.05, Student's t-test) (FIG. 5D). Additionally, γ-VE5 suppressed phosphorylation of Akt at Ser⁴⁷³ without disrupting that at Thr³⁰⁸ (FIG. 5D). Furthermore, the phosphorylation of two Akt downstream targets, murine double minute 2 (MDM2) and inhibitor of nuclear factor kappa-B kinase subunit a (IKKα) was decreased, thus confirming that γ-VE5 inhibited Akt signaling in tumors (FIG. 5D). Similar findings were obtained in LNCaP-abl tumor-bearing mice in which daily treatment with γ-VE5 significantly inhibited tumor growth relative to vehicle-treated controls. As with the PC-3-luc tumors, this suppressive effect on LNCaP-abl tumor growth was also associated with the inhibition of Akt signaling, evident by reduced phosphorylation of Akt at Ser⁴⁷³, MDM2, and IKKα in tumors from γ-VE5-treated mice relative to vehicle-treated controls.

Toxicologic effects of γ-VE5 in PC-3-luc tumor-bearing mice were assessed by body weight, pathologic, and hematologic evaluations. Mean body weights in both vehicle- and γ-VE5-treated groups decreased slightly but insignificantly over the treatment period [−0.2 grams (−0.7%) and −0.7 grams (−2.3%) for vehicle- and γ-VE5-treated, respectively], though the changes in body weight did not differ statistically between the two groups (P=0.35, Student's t-test). Gross pathology findings at necropsy were limited to the presence of abdominal adhesions and variable amounts of clear fluid within the abdomen in γ-VE5-treated mice. Hematological findings after treatment included reductions in hematocrit, red blood cell numbers and hemoglobin concentration in γ-VE5-treated mice relative to controls. Serum chemistry analysis revealed an increase in aspartate aminotransferase and a decrease in serum albumin concentrations in γ-VE5-treated mice. Nonetheless, the values of the affected parameters were within the normal ranges for mice; thus, the clinical relevance of these changes is unclear.

### PH Domain Recognition by Tocopherol and VE5 is Selective for Akt and PHLPP

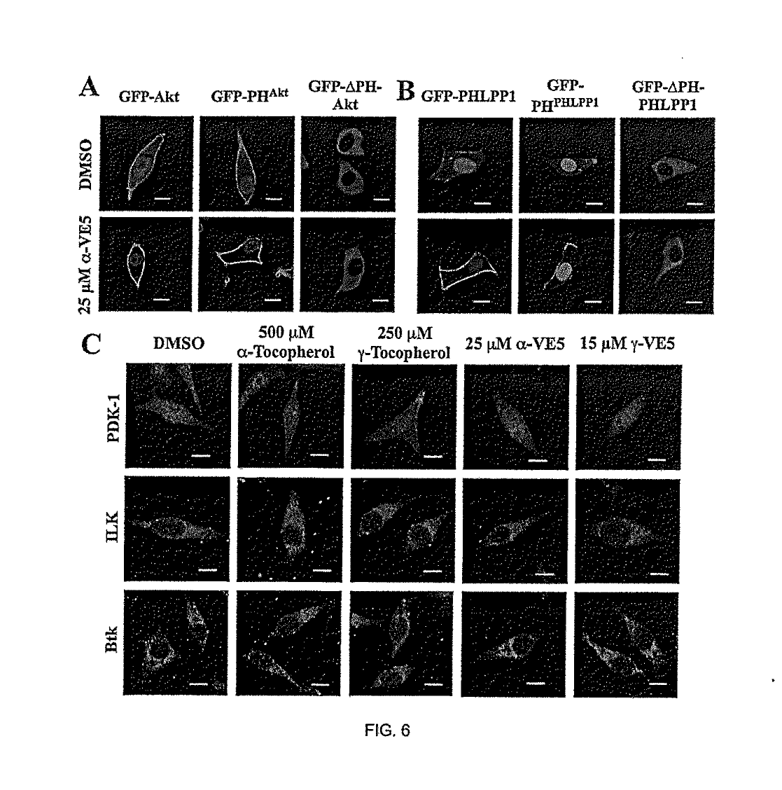

It was hypothesized that the tocopherols and corresponding VE5 derivatives mediated the membrane translocation of Akt and PHLPP through the interaction of their chroman ring head groups with the respective PH domains. To examine this hypothesis, the inventors first investigated the effect of α-VE5 on the intracellular distribution of ectopically expressed GFP-tagged wild-type Akt (GFP-Akt) and Akt-PH domain (PH^{Akt}) compared to PH domain-deleted (ΔPH)-Akt in LNCaP cells. Consistent with the immunocytochemical data (FIG. 4A), GFP-Akt was diffusely distributed in both the cytoplasm and nucleus in vehicle-treated cells, but was localized to the membrane after α-VE5 treatment (FIG. 6A). A similar change in intracellular distribution pattern was also noted in cells expressing the GFP-tagged PH^{Akt} (FIG. 6A). In contrast, the distribution of the ectopically expressed ΔPH-Akt, which appeared to reside exclusively in the cytoplasm, was unaffected by treatment with α-VE5 (FIG. 6A). Similar results were obtained in LNCaP cells ectopically expressing GFP-tagged wild-type PHLPP, PHLPP-PH domain (PH^{PHLPP}), and ΔPH-PHLPP in response to α-VE5 (FIG. 6B). Treatment with α-VE5 led to the membrane localization of cytoplasmic PHLPP and PH^{PHLPP}, whereas the distribution of ΔPH-PHLPP, which appeared to be exclusively cytoplasmic, remained unaffected. Together, these findings indicate that the PH domain is essential to the tocopherol- and VE5-induced translocation of Akt and PHLPP to the cell membrane.

To examine whether this tocopherol- and VE5-induced membrane localization of Akt and PHLPP was selective for these proteins, the impact of these agents on the intracellular distribution of other PH domain-containing kinases, including PDK1, integrin-linked kinase (ILK) (Hannigan et al., Nat Rev Cancer 5, 51 (2005)), and Bruton's tyrosine kinase (BTK), was assessed by immunocytochemistry. No changes were apparent in the distribution of these kinases in response to either tocopherols or VE5 (FIG. 6C), indicating a high degree of selectivity in PH domain recognition by the tocopherols and VE5 compounds at the indicated concentrations.

Next, surface plasmon resonance (SPR) spectroscopy was used to measure the binding affinities of α- and γ-VE5 for the glutathione-S-transferase (GST)-tagged PH domains of Akt and PHLPP compared with those of PDK1 and ILK. Unfortunately, the poor solubility of the tocopherols prohibited their use in SPR analysis because of the formation of oil droplets. For each of the PH domains, the dissociation constants (K_{d}) for α-VE5 and γ-VE5, respectively, were determined (Table 1). These data reveal that the binding affinities of α- and γ-VE5 for the PH domains of Akt and PHLPP paralleled their relative potencies in facilitating dephosphorylation of Ser⁴⁷³ in Akt and apoptosis in LNCaP cells, and were 6- to 29-fold greater than those for the PH domains of PDK1 and ILK. This finding underscores the selectivity of α- and γ-VE5 in facilitating membrane recruitment among PH domain-containing proteins.

TABLE 1
Surface plasmon resonance spectroscopic analysis of the binding affinities
of α- and γ-VE5 for wild-type and mutated PH domains of various
proteins^{a}

K_{d}^{b}

| | α-VE5 | γ-VE5 |
|---|---|---|
| PH domain | (μM) | (μM) |
| Akt | 3.4 ± 0.9 | 0.9 ± 0.1 |
| PHLPP1 | 3.1 ± 0.3 | 0.7 ± 0.1 |
| PDK1 | 19.3 ± 0.6 | 12.4 ± 0.1 |
| ILK | 26.6 ± 1.0 | 18.7 ± 4.5 |
| Akt-Y38G | 0.48 ± 0.13^{c} | 0.49 ± 0.02^{c} |

^{a}K_{d}, dissociation constant; n.d., not determined
^{b}Values represent means (n = 3) ± SD
^{c}P < 0.05 vs. Akt (Student's t-test, n = 3)

To compare the recognition profiles of PIP₃ with those of α- or γ-VE5 for the PH domains of Akt and other PH domain-containing proteins, di-octanoyl PIP₃, a soluble form of PIP₃, was used as ligand for SPR analysis. Of the PH domains tested, PIP₃ exhibited the highest affinity for the PH domain of Akt (K_{d}, 5.4±1.7 nM), consistent with the reported value of 6.2 nM determined by fluorescence resonance energy transfer assays (Ananthanarayanan et al., Proc Natl Acad Sci USA 102, 15081 (2005)), followed by the PH domains of PDK1 (10±3 nM), ILK (16±4 nM), and PHLPP (60±5 nM). These findings show that, while α- and γ-VE5 bound the PH domains of Akt and PHLPP with equal potency, PIP₃ exhibited differential binding to these proteins, because the ratio of the respective IQ values was greater than 10.

Interactions within the PH domain VL2 loop underlie the structural basis for the differential ligand recognition of α-VE5 compared to γ-VE5 by the Akt PH domain

To envisage the mode of ligand recognition between α- or γ-VE5 and the PH domain of Akt, a modeling analysis was carried out using the reported X-ray structure of the PH domain of Akt. Thomas et al., Curr Biol 12, 1256 (2002); Milburn et al., Biochem J 375, 531 (2003). Docking of α- and γ-VE5 into the PH domain suggests that γ-VE5 favored the binding pocket formed within the VL2 loop, in which hydrogen bonding between the OH group of the chroman ring and the peptide backbone of Ala⁵⁰-Pro⁵¹ played a crucial role (FIGS. 7A and B). This γ-VE5 binding motif is distinct from the PIP₃-interacting domain that encompasses the VL1-VL2 loop (FIG. 7A). The involvement of the VL2 loop in γ-VE5 binding was confirmed by the substantial loss of binding affinity for the VL2-deleted mutant of the PH domain determined by SPR (K_{d}, 30±4 μM compared to 0.9±0.1 μM for the wild-type PH domain).

This docking analysis also suggests that the lower binding affinity of α-VE5 (K_{d}, 3.4) relative to γ-VE5 (K_{d}, 0.9 μM) for the Akt PH domain might be attributable to steric factors. Unlike γ-VE5, α-VE5 possesses a methyl group at position 5 of the chroman ring, which might impose steric repulsion with the phenyl ring of the Tyr³⁸ residue (FIG. 7B). To corroborate this hypothesis, we replaced Tyr³⁸ with glycine using site-directed mutagenesis, generating the Y38G PH domain mutant. SPR analysis revealed that this mutation significantly enhanced the binding affinities of α-VE5 and, to a lesser extent, γ-VE5 for the PH domain. The K_{d} values for the binding of α- and γ-VE5 to the Y38G mutant were 0.48±0.13 and 0.49±0.02 μM, respectively, compared to, 3.4±0.2 and 0.9±0.1 μM, respectively, for the wild-type PH domain (P<0.05, n=3 replicates, Student's t-test). Together, these findings support the proposed mode of ligand recognition of α- and γ-VE5 by the Akt PH domain (FIG. 7B).

### Discussion

In this study, evidence was obtained that α- and γ-tocopherol mediate the dephosphorylation of Ser⁴⁷³ in Akt in PTEN-negative LNCaP and PC-3 cells through membrane co-localization with PHLPP. Although tocopherols and PIP₃ share the ability to recruit Akt to the plasma membrane, they lead to opposite effects on Akt functional status due to differences in PH domain recognition. Although it is not thought to be confined exclusively to specific membrane microdomains, PIP₃ can be incorporated into raft microdomains formed in coordination with cholesterol and sphingolipids upon Akt-PH domain binding, where it recruits PDK1 through its PH domain to catalyze phosphorylation of Akt at Thr³⁰⁸. In contrast, these findings show that tocopherols selectively interact with PHLPP to cause dephosphorylation of Akt at Ser⁴⁷³ in non-raft microdomains (FIG. 7C). Indeed, the results suggest that PIP₃- and tocopherol-facilitated Akt recruitment occur in distinct regions (raft or non-raft microdomains) of the cytoplasmic membrane of LNCaP cells, which is consistent with a report that the majority of membrane-associated Akt is present in the phosphorylated form in the non-raft domains of LNCaP cells.

These findings, however, raise the question of how this differential recruitment to distinct membrane regions occurs in light of the three orders of magnitude difference between the binding affinity of PIP₃ and those of α- and γ-tocopherol and α- and γ-VE5 for Akt (nM compared with μM). In this regard, membrane cholesterol content may play an important role. The PIP₃-mediated membrane recruitment and activation of Akt not only involves the complex protein-lipid interaction between Akt and PIP₃, but also requires the coordinated action of cholesterol and sphingolipids to facilitate the formation of raft microdomains. Moreover, reducing the cellular cholesterol content inhibits activation of Akt in lipid rafts. Thus, it is plausible that the suppressive effect of α- and γ-VE5 on the cholesterol content of lipid raft membrane fractions (FIG. 5B) reduced PIP₃-mediated membrane recruitment of Akt to raft microdomains.

In addition, the inventors speculate that PIP₃ might exhibit differential binding affinities for dephosphorylated and phosphorylated forms of Akt. Despite elevated PIP₃ abundance in phosphatase and tensin homolog (PTE/V)-negative LNCaP cells, the majority of Akt resided in the cytoplasm (FIGS. 1D and 4A), which is in line with the general notion that Akt, once phosphorylated, is released from the membrane to interact with and phosphorylate target proteins in the cytoplasm and nucleus. Nevertheless, this cytoplasmic pool of phosphorylated Akt was responsive to tocopherol- or VE5-induced membrane localization to non-raft domains and consequent dephosphorylation. This possible difference in the mode of recognition of Akt by tocopherols or VE5 compared to PIP₃, along with the aforementioned reductions in cholesterol content of raft microdomains, may underlie the net dephosphorylation of Akt in tocopherol or VE5-treated cells.

In light of the tumor suppressor role of PHLPP in blocking PTEN-mutant prostate cancer progression (Chen et al., Cancer Cell 20, 173 (2011)) and in mediating androgen receptor-induced inhibition of Akt (Carver et al., Cancer Cell 19, 575 (2011); Mulholland et al., Cancer Cell 19, 792 (2011)) PHLPP activation represents a therapeutically relevant target for prostate cancer. However, from a chemopreventive perspective, the high concentrations required for α- and γ-tocopherol to induce PHLPP-mediated Akt inhibition (greater than 150 μM) might not be attainable in humans through vitamin E supplementation. The human diet provides mainly α- and γ-tocopherols, whereas supplements generally supply vitamin E as α-tocopherol acetate in a racemic form (all-rac-α-tocopherol acetate). Due to its hydrophobic nature, the intestinal absorption, plasma transport, and cellular uptake of vitamin E require protein-mediated processes that involve transporters, such as the scavenger receptor class B type I, and various plasma lipoproteins, which might represent a limiting factor for achieving high plasma concentrations of vitamin E. For example, vitamin E concentration in the plasma reaches a plateau at 600 mg of daily supplementation. In light of the 10-fold higher potency, a or γ-VE5 provides a proof-of-concept that PHLPP is a “druggable” target, which is supported by the in vivo efficacy of γ-VE5 in suppressing the growth of PC-3 and LNCaP-abl xenograft tumors in athymic nude mice (FIG. 5D).

Hydrophobicity of the long aliphatic side chains of the tocopherols may limit their access to the PH domains of Akt and PHLPP at the membrane-cytoplasm interface. Thus, truncation of the side chains by two isopranyl units endowed α- and γ-VE5 with a more favorable physicochemical property compared to α- and γ-tocopherol for the membrane recruitment of the target proteins, resulting in greater dephosphorylation of Akt. The membrane-targeting of PHLPP and its dephosphorylation of Akt depends on the protein Scrib, which facilitates formation of a PHLPP-Akt-Scrib heterotrimeric complex at the cell membrane. Li et al., EMBO Rep 12, 818 (2011). Because Scrib deficiency contributes to prostate tumorigenesis in preclinical models and its deregulation is associated with poor prognosis in human prostate cancer patients (Pearson et al., J Clin Invest 121, 4257 (2011)), the use of PH domain-targeted agents, like α- and γ-VE5, may yield therapeutic benefits by restoring PHLPP-mediated dephosphorylation of Akt in prostate tumors with dysfunctional Scrib.

Consistent with predicted activity against tumors with activated Akt status, γ-VE5 as a single agent displayed strong tumor-suppressive activity in vivo against the growth of subcutaneous xenograft tumors established from the PTEN-null PC-3 and LNCaP-abl prostate cancer cell lines. The abdominal adhesions and ascites in γ-VE5-treated mice were possibly a response to chronic irritation and peritonitis associated with repeated daily intraperitoneal injections of the compound, and may underlie the mild weight loss observed in these mice. Although complete blood counts revealed indications of anemia, the clinical relevance of these findings is unclear as these values were all within normal limits for mice. Similarly, the affected concentrations of aspartate aminotransferase and albumin were also within normal limits and were not associated with changes in other indicators of liver function. Studies designed to more thoroughly examine the toxicopathological effects of this novel class of compounds are needed to more completely evaluate their translational potential.

The SPR analysis showed that α- and γ-VE5 exhibited selectivity in binding affinity and membrane recruitment for Akt and PHLPP compared to other PH domain-containing proteins, such as PDK-1 and ILK. However, this selectivity was moderate, as estimated by the ratios of the K_{d} values, which ranged from 6 to 29, whereas immunocytochemical data revealed a difference in the membrane recruitment of the higher affinity (Akt, PHLPP) compared to the lower affinity (PDK-1, ILK) PH domain-containing proteins in response to α- and γ-VE5 treatment (FIG. 4 and FIG. 6C). Because α- and γ-VE5 passively diffuse into the plasma membrane, the local concentration at the membrane-cytoplasm interface becomes a limiting factor that controls the recruitment of PH domain-containing proteins from the cytoplasm. For instance, γ-VE5 at 15 μM presumably achieves local concentrations at the membrane high enough to facilitate the localization of Akt and PHLPP (FIG. 4), but that are insufficient for the recruitment of the low-affinity PDK1 and ILK (FIG. 6C). This concentration-dependent control of membrane recruitment was corroborated by the time-dependent increase in membrane localization of PDK-1 in response to higher concentrations of γ-VE5. At 25 μM and 35 μM of γ-VE5, membrane-associated PDK-1 accounted for approximately 50% and greater than 90%, respectively, of the total immunofluorescence signal for PDK-1 after 6 hours of exposure (longer exposure led to cell detachment).

The preferential binding of α- and γ-VE5 to the PH domain of Akt relative to those of PDK-1, ILK, and BTK is intriguing. Each PH domain contains a sequence homologous to that of the VL2 loop of the PH domain of Akt. However, conformational analysis revealed differences in the secondary structures between the Akt VL2 loop and these other sequences. Specifically, the PH domains of PDK1, BTK, and ILK contain β-sheet structures in lieu of a variable loop structure. Thus, this difference in their secondary structures might underlie the ability of tocopherol and the VE5 derivatives to discriminate between Akt and other PH-domain containing proteins.

In summary, this study describes a mechanism by which vitamin E mediates dephosphorylation of Ser⁴⁷³ in Akt in cancer cells. This mode of Akt inhibition is different from that of kinase inhibitors or PH domain-targeted inhibitors and has several implications. Barnett et al., Biochem J 385, 399 (2005); Moses et al., Cancer Res 69, 5073 (2009). First, it provides a rationale for the pharmacological exploitation of vitamin E to develop a novel class of Akt inhibitors, of which the proof-of-principle is provided by α- and γ-VE5, through side-chain truncation. Second, PTEN mutations, a common genetic aberration in various hereditary and sporadic cancers lead to PIP₃ accumulation and subsequent Akt activation. Salmena et al., Cell 133, 403 (2008). The finding in LNCaP cells suggests the ability of tocopherols and VE5 to counteract Akt activation secondary to defects such as loss of PTEN function or Scrib dysregulation. Third, targeting the PH domains of Akt and PHLPP to facilitate their membrane translocation represents a new concept for developing Akt inhibitors, which warrants investigation.

### Example 2

### Synthesis and Activity of Tocopheryl Derivatives

A number of the compounds prepared are shown below, along with their designation and in some cases their ability to inhibit proliferation of LNCaP and/or PC-3 cells.

| | | IC₅₀ (μM)* | |
|---|---|---|---|
| Cpd | Structure | LNCaP | PC-3 |
| γ-VE5 | 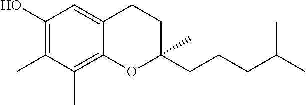 [SMILES: Cc1c(O)cc2c(c1C)O[C@](C)(CCCC(C)C)CC2] (R)-2,7,8-trimethyl-2-(4-methylpentyl)- chroman-6-ol | 12^{§} | 8^{§} |
| α-VE5 | 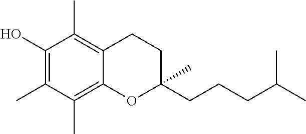 [SMILES: Cc1c(C)c2c(c(C)c1O)CC[C@@](C)(CCCC(C)C)O2] (R)-2,5,7,8-tetramethyl-2-(4-methylpentyl)- chroman-6-ol | 20^{§} | 15^{§} |
| rac-VE5 | 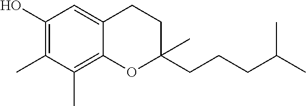 [SMILES: Cc1c(O)cc2c(c1C)OC(C)(CCCC(C)C)CC2] | 20 | — |
| 1 | 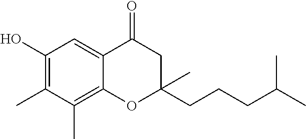 [SMILES: Cc1c(O)cc2c(c1C)OC(C)(CCCC(C)C)CC2=O] 6-hydroxy-2,7,8-trimethyl-2-(4-methyl- pentyl)chroman-4-one | 7 | 5 |
| 2 | 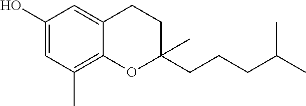 [SMILES: Cc1cc(O)cc2c1OC(C)(CCCC(C)C)CC2] 2,8-dimethyl-2-(4-methylpentyl)chroman-6-ol | — | 9 |
| 3 | 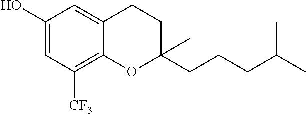 [SMILES: CC(C)CCCC1(C)CCc2cc(O)cc(C(F)(F)F)c2O1] 2-methyl-2-(4-methylpentyl)-8-(trifluoro- methyl)chroman-6-ol | — | 8 |
| 4 | 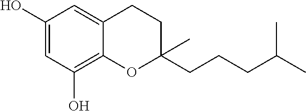 [SMILES: CC(C)CCCC1(C)CCc2cc(O)cc(O)c2O1] 2-methyl-2-(4-methylpentyl)chroman-6,8-diol | — | 27 |
| 5 | 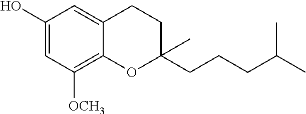 [SMILES: COc1cc(O)cc2c1OC(C)(CCCC(C)C)CC2] 8-methoxy-2-methyl-2-(4-methylpentyl)- chroman-6-ol | — | 28 |
| 6 | 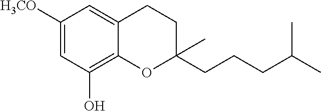 [SMILES: COc1cc(O)c2c(c1)CCC(C)(CCCC(C)C)O2] 3-methoxy-7-methyl-7-(4-methylpentyl)- 5,6,7,8-tetrahydronaphthalen-1-ol | — | >30 |
| 7 | 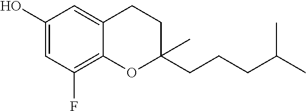 [SMILES: CC(C)CCCC1(C)CCc2cc(O)cc(F)c2O1] 8-fluoro-2-methyl-2-(4-methylpentyl)- chroman-6-ol | — | 7 |
| 8 | 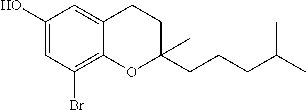 [SMILES: CC(C)CCCC1(C)CCc2cc(O)cc(Br)c2O1] 8-bromo-2-methyl-2-(4-methylpentyl)- chroman-6-ol | — | 7 |
| 9 | 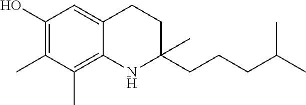 [SMILES: Cc1c(O)cc2c(c1C)NC(C)(CCCC(C)C)CC2] 2,7,8-trimethyl-2-(4-methylpentyl)-1,2,3,4- tetrahydroquinolin-6-ol | — | 15 |
| 10 | 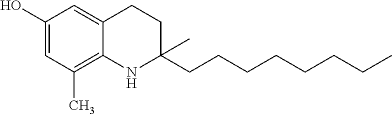 [SMILES: CCCCCCCCC1(C)CCc2cc(O)cc(C)c2N1] 2,8-dimethyl-2-octyl-1,2,3,4-tetrahydro- quinolin-6-ol | — | 8.5 |
| 11 | 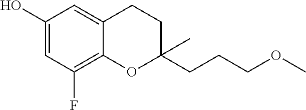 [SMILES: COCCCC1(C)CCc2cc(O)cc(F)c2O1] 8-fluoro-2-(3-methoxypropyl)-2-methyl- chroman-6-ol | — | >30 |
| 12 | 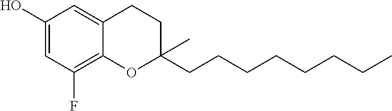 [SMILES: CCCCCCCCC1(C)CCc2cc(O)cc(F)c2O1] 8-fluoro-2-methyl-2-octylchroman-6-ol | — | 8.5 |
| 13 | 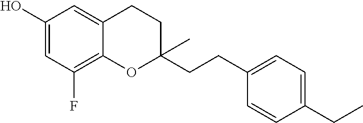 [SMILES: CCc1ccc(CCC2(C)CCc3cc(O)cc(F)c3O2)cc1] 2-(4-ethylphenethyl)-8-fluoro-2-methyl- chroman-6-ol | — | 9 |
| 14 | 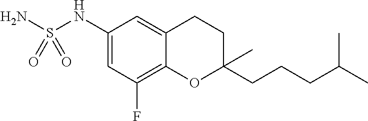 [SMILES: CC(C)CCCC1(C)CCc2cc(NS(N)(=O)=O)cc(F)c2O1] | — | — |
| 15 | 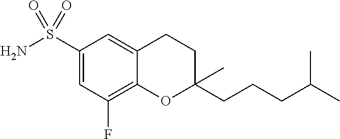 [SMILES: CC(C)CCCC1(C)CCc2cc(S(N)(=O)=O)cc(F)c2O1] 8-fluoro-2-methyl-2-(4-methylpentyl)- chroman-6-sulfonamide | — | — |
| 16 | 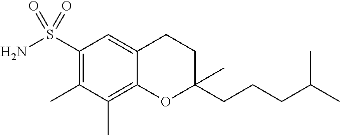 [SMILES: Cc1c(S(N)(=O)=O)cc2c(c1C)OC(C)(CCCC(C)C)CC2] 2,7,8-trimethyl-2-(4-methylpentyl)-chroman- 6-sulfonamide | — | — |
| 17 | 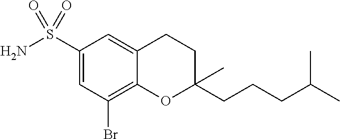 [SMILES: CC(C)CCCC1(C)CCc2cc(S(N)(=O)=O)cc(Br)c2O1] 8-bromo-2-methyl-2-(4-methylpentyl)- chroman-6-sulfonamide | — | — |
| 18 | 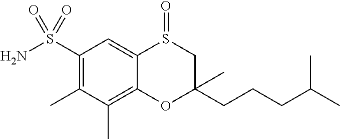 [SMILES: Cc1c(S(N)(=O)=O)cc2c(c1C)OC(C)(CCCC(C)C)CS2=O] 2,7,8-trimethyl-2-(4-methylpentyl)-2,3- dihydrobenzo[b][1,4]oxathiine-6-sulfonamide 4-oxide | — | — |
| 19 | 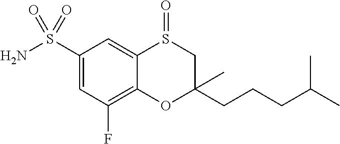 [SMILES: CC(C)CCCC1(C)CS(=O)c2cc(S(N)(=O)=O)cc(F)c2O1] 8-fluoro-2-methyl-2-(4-methylpentyl)-2,3- dihydrobenzo[b][1,4]oxathiine-6-sulfonamide 4-oxide | — | — |
| 20 | 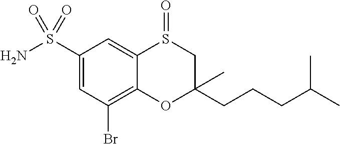 [SMILES: CC(C)CCCC1(C)CS(=O)c2cc(S(N)(=O)=O)cc(Br)c2O1] 8-bromo-2-methyl-2-(4-methylpentyl)-2,3- dihydrobenzo[b][1,4]oxathiine-6-sulfonamide 4-oxide | — | — |
| 21 | 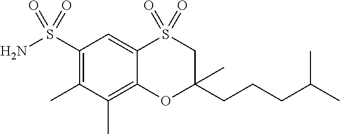 [SMILES: Cc1c(S(N)(=O)=O)cc2c(c1C)OC(C)(CCCC(C)C)CS2(=O)=O] 2,7,8-trimethyl-2-(4-methylpentyl)-2,3- dihydrobenzo[b][1,4]oxathiine-6-sulfonamide 4,4-dioxide | — | — |
| 22 | 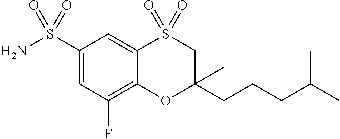 [SMILES: CC(C)CCCC1(C)CS(=O)(=O)c2cc(S(N)(=O)=O)cc(F)c2O1] 8-fluoro-2-methyl-2-(4-methylpentyl)-2,3- dihydrobenzo[b][1,4]oxathiine-6-sulfonamide 4,4-dioxide | — | — |
| 23 | 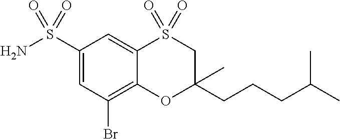 [SMILES: CC(C)CCCC1(C)CS(=O)(=O)c2cc(S(N)(=O)=O)cc(Br)c2O1] 8-bromo-2-methyl-2-(4-methylpentyl)-2,3- dihydrobenzo[b][1,4]oxathiine-6-sulfonamide 4,4-dioxide | — | — |
| 24 | 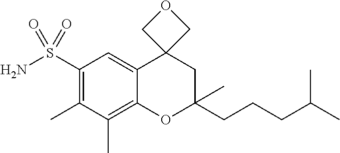 [SMILES: Cc1c(S(N)(=O)=O)cc2c(c1C)OC(C)(CCCC(C)C)CC21COC1] 2,7,8-trimethyl-2-(4-methylpentyl)- spiro[chroman-4,3′-oxetane]-6-sulfonamide | — | — |
| 25 | 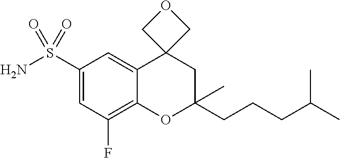 [SMILES: CC(C)CCCC1(C)CC2(COC2)c2cc(S(N)(=O)=O)cc(F)c2O1] 8-fluoro-2-methyl-2-(4-methylpentyl)- spiro[chroman-4,3′-oxetan]-6-sulfonamide | — | — |
| 26 | 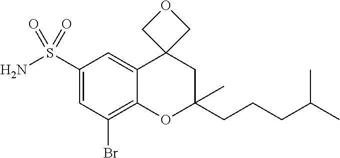 [SMILES: CC(C)CCCC1(C)CC2(COC2)c2cc(S(N)(=O)=O)cc(Br)c2O1] 8-bromo-2-methyl-2-(4-methylpentyl)- spiro[chroman-4,3′-oxetane]-6-sulfonamide | — | — |
| 27 | 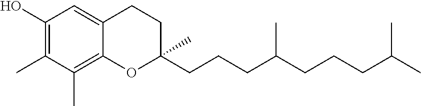 [SMILES: Cc1c(O)cc2c(c1C)O[C@](C)(CCCC(C)CCCC(C)C)CC2] (2R)-2-(4,8-dimethylnonyl)-2,7,8- trimethylchroman-6-ol | 30 | — |
| 28 | 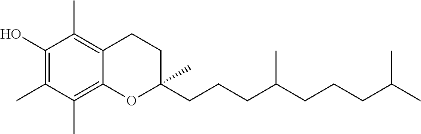 [SMILES: Cc1c(C)c2c(c(C)c1O)CC[C@@](C)(CCCC(C)CCCC(C)C)O2] (2R)-2-(4,8-dimethylnonyl)-2,5,7,8- tetramethylchroman-6-ol | 42 | — |
| 29 | 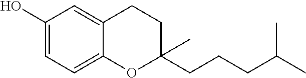 [SMILES: CC(C)CCCC1(C)CCc2cc(O)ccc2O1] 2-methyl-2-(4-methylpentyl)chroman-6-ol | 40 | — |
| 30 | 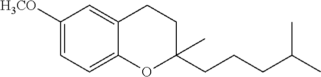 [SMILES: COc1ccc2c(c1)CCC(C)(CCCC(C)C)O2] 6-methoxy-2-methyl-2-(4-methylpentyl)- chroman | 40 | — |
| 31 | 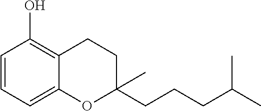 [SMILES: CC(C)CCCC1(C)CCc2c(O)cccc2O1] 2-methyl-2-(4-methylpentyl)chroman-5-ol | 18 | — |
| 32 | 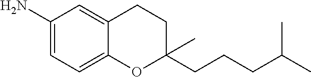 [SMILES: CC(C)CCCC1(C)CCc2cc(N)ccc2O1] 2-methyl-2-(4-methylpentyl)chroman-6- amine | >50 | — |
| 33 | 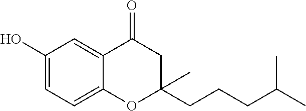 [SMILES: CC(C)CCCC1(C)CC(=O)c2cc(O)ccc2O1] 6-hydroxy-2-methyl-2-(4- methylpentyl)chroman-4-one | 30 | — |
| 34 | 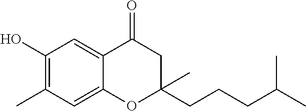 [SMILES: Cc1cc2c(cc1O)C(=O)CC(C)(CCCC(C)C)O2] 6-hydroxy-2,7-dimethyl-2-(4- methylpentyl)chroman-4-one | 30 | — |
| 35 | 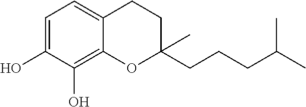 [SMILES: CC(C)CCCC1(C)CCc2ccc(O)c(O)c2O1] 2-methyl-2-(4-methylpentyl)chroman-7,8-diol | >50 | — |
| 36 | 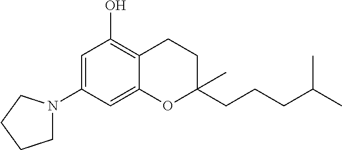 [SMILES: CC(C)CCCC1(C)CCc2c(O)cc(N3CCCC3)cc2O1] 2-methyl-2-(4-methylpentyl)-7-(pyrrolidin-1- yl)chroman-5-ol | 21 | — |
| 37 | 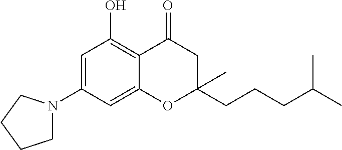 [SMILES: CC(C)CCCC1(C)CC(=O)c2c(O)cc(N3CCCC3)cc2O1] 5-hydroxy-2-methyl-2-(4-methylpentyl)-7- (pyrrolidin-1-yl)chroman-4-one | >50 | — |

*1% FBS-containing or serum-free RPMI 1640 medium for 24 h
^{§}Please see the attached manuscript for detailed information and their mode of action of γ- and α-VE5.

TABLE 2
Antiproliferative potencies of compound 1 in different cancer cell lines

| | IC₅₀ (μM) | | | | |
|---|---|---|---|---|---|
| FBS (%) | LNCaP | PC-3 | AsPC-1 | BxPC-3 | MDA-MB-231 |
| 0 | — | 4 | 5 | 7 | 6.5 |
| 1 | 9 | 7 | 11 | 11 | — |
| 5 | >20 | 14 | 18 | 21 | 20 |

### Synthetic Procedures

A. Synthesis of optically active γ- and α-VE5 (compounds 1 and 2). See FIG. 8 for an overview of the synthesis.

### Experimental Procedures

Step a:

To a suspension of appropriate Wittig ylied (Ph₃PCH₂CH₂CH(CH₃)₂Br) (22 mmol) in anhydrous THF (100 mL) at 0° C. was added lithiumbis(trimethylsilyl)amide (22 mL, 1M solution in THF) dropwise. After 30 min. a solution of benzoic acid 2-formyl-2,5,7,8-tetramethyl-chroman-6-yl ester (1; 15 mmol) in anhydrous THF was added dropwise within 15 min. The reaction mixture was allowed to attain rt and further stirred for 3 h. The reaction mixture was diluted with water (300 mL) and extracted with ethyl acetate. The organic layer was dried over anhydrous Na₂SO₄ and evaporated to dryness. The oily residue was purified by column chromatography using hexane as an eluent to give the desired product ii. Yield 80-95%.

Step b:

Compound 1i (13 mmol) was dissolved in ethyl acetate (100 mL) and hydrogenated overnight at 35 psi using 10% Pd/C (500 mg) as catalyst to give γ-VE5 in 95-98% yield.

Step c:

γ-VE5 (13.35 mmol) in hexane (100 mL) was added dropwise a solution of Br₂ (14 mmol) in hexane (25 mL) at rt. The reaction mixture was further stirred for 3.5 h and evaporated to dryness. The residue were dissolved in DCM (100 mL) and treated with AcOH (48 mL), Ac₂O (8.8 mL) and H₂SO₄ (1 mL). The mixture was stirred at rt for overnight. The solvents were removed under vacuo and the residue was extracted with ethyl acetate, washed successively with water and brine. The organic layer was dried over anhy.Na₂SO₄ and evaporated to dryness to give the bromo-derivative iii in 80-95% yield.

Step d:

To a solution of compound iii (9 mmol) in anhyd. acetonitrile (30 mL) was added NMPNO (36 mmol) and the mixture was stirred for 18 h at rt. The reaction mixture was concentrated to 1/3 and poured into water and extracted with ethylacetate. The organic layer washed successively with 5% HCl, water, brine and evaporated to dryness to give the corresponding aldehyde iv in 85-90% yield.

Step 2:

To a solution of compound 1v (9 mmol), in dioxane (120 mL), NH₂SO₃H (14.4 mmol) and water (40 mL) were added. After stirring for 20 min, NaClO₂ (12.6 mmol) and water (30 mL) were added and the reaction mixture was further stirred for 30 min. The reaction mixture was then diluted with water, extracted with ethyl acetate (3×200 mL). The combined organic fractions were washed with water, dried over anhy. Na₂SO₄ and evaporated to dryness to furnish compound v in 70-85% yield.

Step f:

A solution of compound v (8 mmol) in MeOH and Con. HCl (15 mL) was refluxed for 2 h. 500 mL water was added and the reaction mixture was extracted with ethyl acetate, dried over anhy. Na₂SO₄ and evaporated to dryness to yield the hydroxyl acid vi in 90-95% yield.

Step g:

Compound vi was heated at 170° C. for 3 h and cooled to rt. The desired product was purified by column chromatography to give γ-VE5 in 50-70% yield.

B. Synthesis of optically active compounds 27 and 28 is similar to the aforementioned procedure except the use of the longer aliphatic side chain in the Wittig reaction described in step a.

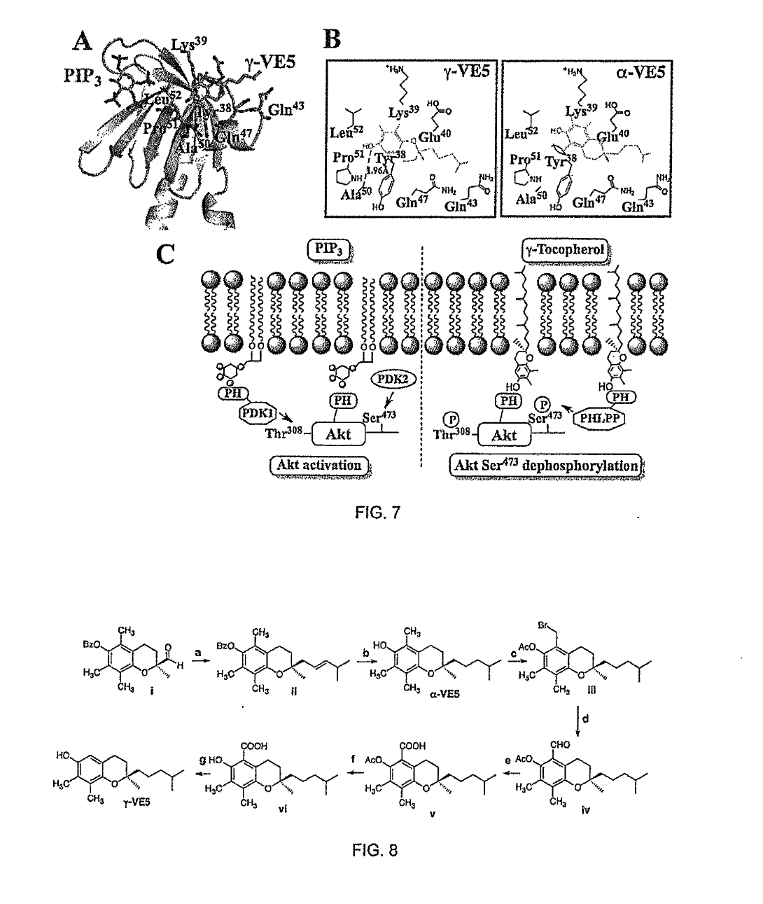

C. Synthesis of compounds 3, and 29-37 (racemic). See FIG. 9 for an overview of the synthesis.

### General Procedure:

Step a:

In a dean-stark distillation assembly, a mixture of appropriate 2-hydroxy acetophenone (50 mmol), 6-methyl-heptan-2-one (51 mmol) and pyrrolidine (50 mmol) in anhydrous toluene (250 mL) was stirred and heated to reflux for 8-20 h. After completion of reaction, the solvent was removed under reduced pressure and the residue was extracted with EA and washed with 1N HCl (200 mL) followed by brine (200 mL). The organic phase was separated, dried over anh. Na₂SO₄ and the residue was purified by column chromatography using Hexane/ethyl acetate as an eluent to give chromone in 40-80% yield.

Step b:

The appropriate chromone (2 mmol) was dissolve in MeOH and carefully treated with Zn (20 mmol) and con. HCl (50 mmol) at rt. The reaction mixture was stirred at rt for 5 h and filtered through celite. The filtrate was evaporated to dryness and the crude product was purified by column chromatography using ethyl acetate/hexane as an eluent to furnish corresponding tocopherol derivatives in 50-70% yield.

### Example III

### Additional Structure-Activity Studies

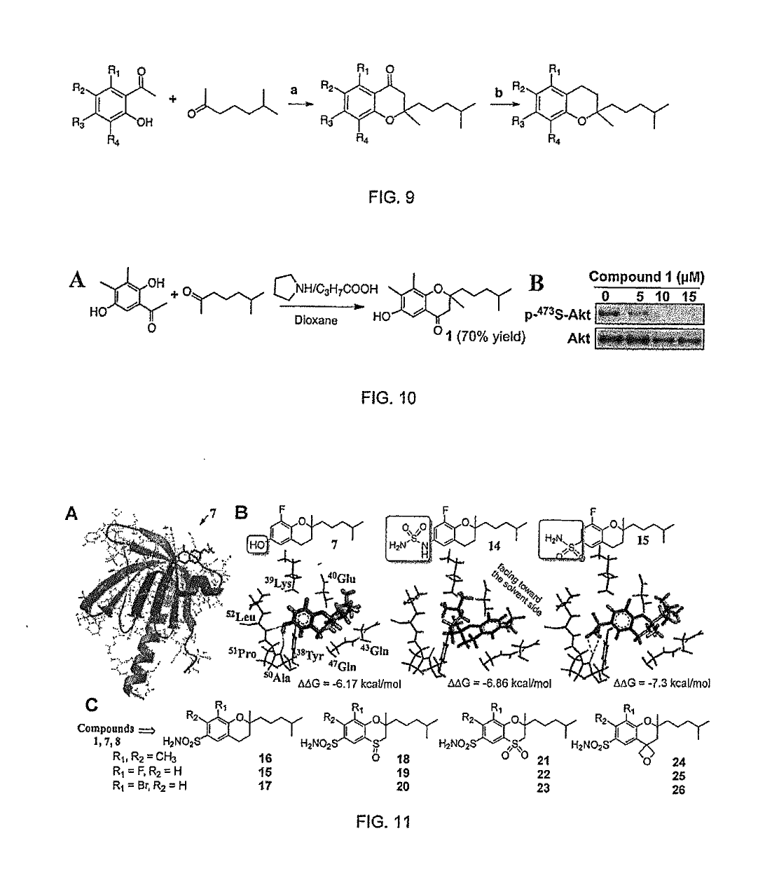

Structure-activity relationship (SAR) of tocopheryl derivatives. Modeling analysis suggests that γ-VE5 interacts with the V2 loop of the Akt PH domain through hydrogen bonding of its polar head group with the 50Ala-51Pro peptide backbone (FIG. 7A and FIG. 7B). From a structural perspective, this binding affinity might be improved by increasing polar interactions of the ligand with the hydrophilic residues within the binding pocket. The proof-of-concept of this premise was obtained by compound 1 (FIG. 10A), in which the C-4 of the chroman ring was converted to a keto function. Compound 1 was more potent than γ-VE5 in binding to the Akt PH domain (Table 3, Kd) and in suppressing Ser-473 Akt phosphorylation (FIG. 10B) and cell viability (Table 3, IC₅₀). However, compound I lacked in vivo efficacy against PC-3 xenograft tumor growth, which might be attributable to metabolic instability, presumably via reduction of the keto function coupled with glucuronidation. The inventors propose, however, that this problem might be circumvented through isosteric replacement, as described further herein.

To further refine the working hypothesis, a series of γ-VE5 derivatives (FIG. 11) were synthesized for SAR analysis by interrogating their binding affinities to the Akt PH domain (K_{d}) versus antitumor activities against PC-3 cells (IC₅₀), of which the data are summarized in Table 3.

TABLE 3
SAR of γ-VE5 versus compounds 1-9

| | | | | | | | | | | γ-VE5 |
|---|---|---|---|---|---|---|---|---|---|---|
| 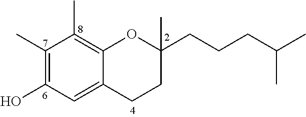 [SMILES: Cc1c(O)cc2c(c1C)OC(C)(CCCC(C)C)CC2] (the numbers denote C positions) | | | | | | | | | | |
| | | | | | | | | | | 1 |
| 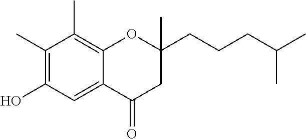 [SMILES: Cc1c(O)cc2c(c1C)OC(C)(CCCC(C)C)CC2=O] | | | | | | | | | | |
| | | | | | | | | | | 2-9 |
| 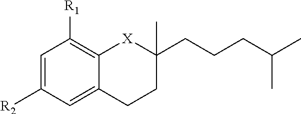 | | | | | | | | | | |
| Compound | γ-VE5 | 1 | 2 | 3 | 4 | 5 | 6 | 7 | 8 | 9 |
| X | Structures | | O | O | O | O | O | O | O | N |
| R1 | shown | | —CH₃ | —CF₃ | —OH | —OCH₃ | —OH | —F | —Br | —CH₃ |
| R2 | above | | —OH | —OH | —OH | —OH | —OCH₃ | —OH | —OH | —OH |
| K_{d} (μM) | 0.9 | 0.32 | 0.6 | 0.7 | 2.0 | 3.1 | 11 | 0.5 | 0.65 | 0.1 |
| IC₅₀ (μM) | 8.5 | 5 | 9 | 8 | 27 | 28 | >30 | 7 | 7 | 15 |

As the methyl function at C-7 does not play a determining role in ligand binding, γ-VE5 can be structurally simplified to its δ-counterpart (i.e., compound 2), providing a scaffold for modification (compounds 3-9). With the exception of compound 9, there is a correlation between K_{d} and IC₅₀ values, suggesting a causal relationship between Akt PH domain binding and antiproliferative activity among these derivatives. It is plausible that compound 9, despite its tight binding to the PH domain, could not be effectively incorporated into the negatively charged inner leaflet of the cytoplasmic membrane due to the electron-rich amino substituent, thereby diminishing its antiproliferative efficacy. This premise was supported by the ability to enhance the antitumor potency of compound 9 by increasing its aliphatic side chain length (Table 4, compound 10), suggesting interplay between the head group and aliphatic side chain in determining the antiproliferative potency presumably by affecting membrane association. For example, insertion of an oxygen atom into the side chain of compound 7 abrogated its antiproliferative activity. In contrast, increasing the chain length or inserting an aromatic ring in compound 7 exerted no substantial changes in the IC₅₀ values (Table 4).

TABLE 4
Effect of Chain Length on Antiproliferative Activity

| | |
|---|---|
| | 10 |
| 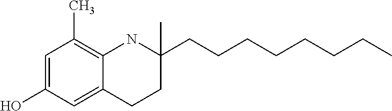 [SMILES: CCCCCCCCC1(C)CCc2cc(O)cc(C)c2N1] | |
| | 11 |
| 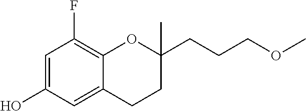 [SMILES: COCCCC1(C)CCc2cc(O)cc(F)c2O1] | |
| | 12 |
| 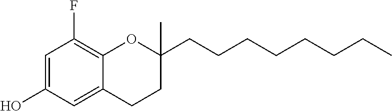 [SMILES: CCCCCCCCC1(C)CCc2cc(O)cc(F)c2O1] | |
| | 13 |
| 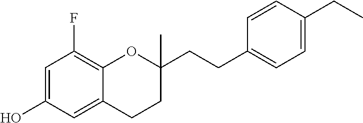 [SMILES: CCc1ccc(CCC2(C)CCc3cc(O)cc(F)c3O2)cc1] | |

| Compounds | 10 | 11 | 12 | 13 |
|---|---|---|---|---|
| IC₅₀ (μM) | 8.5 | >30 | 8.5 | 9 |

Lead optimization through isosteric replacement. Isosteres are groups which have chemical and physical similarities producing broadly similar biological effects. The above SAR analysis identified compounds 1, 7, and 8 as candidates for continued modifications with the goal of generating 2^{nd}-generation PHLPP1-targeted Akt inhibitors with increased antitumor potency and metabolic stability. In light of the improved in vitro antiproliferative activity of compound 1 relative to γ-VE5, it will be desirable to introduce a 4-keto function into both compounds 7 and 8. However, this keto function, as well as the phenolic —OH are intrinsically metabolically unstable because of their susceptibility to metabolic transformations, leading to loss of potency and/or rapid excretion. Substantial evidence indicates that bioisosteric replacement represents a successful strategy to enhance potency and/or to improve pharmacokinetic properties. As the identification of an effective isostere is highly context-specific, modeling analysis was employed to aid isostere selection. For example, although both sulfamide (—NHSO₂NH₂) and sulfonamide (—SO₂NH₂) are commonly used as isosteres for phenolic —OH, docking of compound 7 vis-à-vis its sulfamide (14) and sulfonamide (15) counterparts revealed different modes of binding inside the Akt PH domain (FIGS. 10A & B). While the sulfonamide derivative (15) mimics the binding of compound 7 with higher affinity (ΔΔG, −7.3 versus −6.17 kcal/mol) (FIG. 10B), the sulfamide analogue adopts a different binding mode, as the polar head group is oriented toward an opposite direction. In light of this finding, the inventors propose to use sulfonamide to replace the phenolic —OH in the structural optimization of compounds 1, 7, and 8.

For the replacement of the 4-keto function, sulfone (—SO), sulfoxide (—SO₂), and oxetane moieties have been commonly probed as ketone isosteres. Modeling analysis indicates that replacement of 4-keto with any of these three isosteres would not give rise to changes in the mode of interaction with the Akt PH domain. Accordingly, the proposed isosteric replacements of the phenolic —OH and 4-keto will generate a total of twelve 2nd-generation γ-VE5 derivatives (FIG. 10C).

The complete disclosure of all patents, patent applications, and publications, and electronically available materials cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A compound according to formula I: 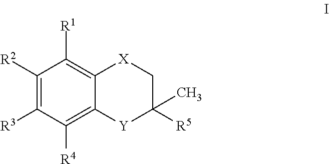
wherein R¹, R², R³ and R⁴ are selected from H, CH₃, OH, SH, OCH₃, NHR′, halogen, CF₃, N-linked pyrrolidine, and SO₂NHR′, or any combination thereof;
R⁵ is an alkyl, alkenyl, or alkaryl group including from 4 to 11 carbons,
X is selected from CH₂, CHOH, C═O, S═O, O═S═O, and an oxetane ring,
Y is selected from CH₂, O, and NH, and
R′ is a H, aryl, or a lower alkyl group, or pharmaceutically acceptable salts thereof.

2. The compound according to claim 1, wherein R⁵ is an alkyl group having from 4 to 6 carbons.

3. The compound according to claim 1, wherein R⁵ is an alkyl, alkenyl, or alkaryl group having from 7 to 11 carbons.

4. The compound according to claim 1, wherein Y is an O.

5. The compound according to claim 1, wherein R¹ and R³ are H.

6. The compound according to claim 1, wherein R² is OH.

7. The compound according to claim 1, wherein X is selected from C═O, S═O, and O═S═O

8. The compound according to claim 7, wherein X is C═O.

9. The compound according to claim 1, wherein R² is OH, X is C═O, and Y is O.

10. The compound according to claim 1, wherein the compound is selected from the following structures:

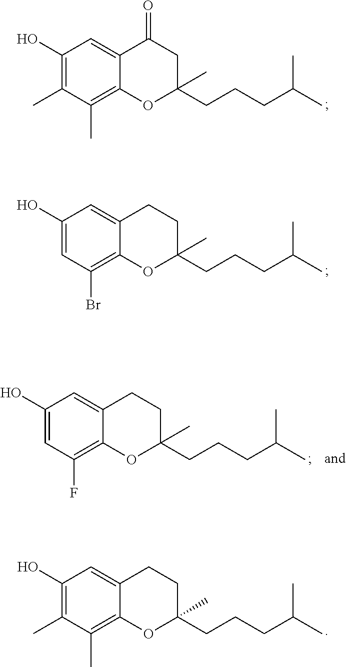 [SMILES: Cc1c(O)cc2c(c1C)OC(C)(CCCC(C)C)CC2=O ; CC(C)CCCC1(C)CCc2cc(O)cc(Br)c2O1 ; CC(C)CCCC1(C)CCc2cc(O)cc(F)c2O1 ; Cc1c(O)cc2c(c1C)O[C@](C)(CCCC(C)C)CC2]

11. A method of treating cancer in a subject in need thereof, comprising administering to the subject a therapeutically effective amount of a compound according to Formula I: 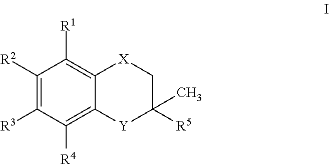
wherein R¹, R², R³ and R⁴ are selected from H, CH₃, OH, SH, OCH₃, NHR′, halogen, CF₃, N-linked pyrrolidine, and SO₂NHR′, or any combination thereof;
R⁵ is an alkyl, alkenyl, or alkaryl group including from 4 to 11 carbons,
X is selected from CH₂, CHOH, C═O, S═O, O═S═O, and an oxetane ring,
Y is selected from CH₂, O, and NH, and
R′ is a H, aryl, or a lower alkyl group, or a pharmaceutically acceptable salt thereof.

12. The method of claim 11, wherein the cancer is a cancer involving Akt signaling dysregulation.

13. The method of claim 11, wherein the cancer is prostate cancer.

14. The method of claim 11, wherein the compound is administered in a pharmaceutically acceptable carrier.

15. The method according to claim 11, wherein R² of the compound of formula I is OH.

16. The method according to claim 11, wherein X of the compound of formula I is selected from C═O, S═O, and O═S═O.

17. The method according to claim 11, wherein the compound of formula I is further defined such that R² is OH, X is C═O, and Y is O.

18. A method of inactivating Akt in a tumor cell by contacting the tumor cell with an effective amount of a compound according to formula I: 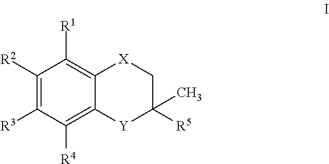
wherein R¹, R², R³ and R⁴ are selected from H, CH₃, OH, SH, OCH₃, NHR′, halogen, CF₃, N-linked pyrrolidine, and SO₂NHR′, or any combination thereof;
R⁵ is an alkyl, alkenyl, or alkaryl group including from 4 to 11 carbons,
X is selected from CH₂, CHOH, C═O, S═O, O═S═O, and an oxetane ring,
Y is selected from CH₂, O, and NH, and
R′ is a H, aryl, or a lower alkyl group, or a pharmaceutically acceptable salt thereof.

19. The method of claim 18, wherein Akt is inactivated while present in the membrane of the tumor cell.

20. The method of claim 18, wherein the tumor cell is in vitro.
